(19) United States
(12) Patent Application Publication
(10) Pub. No.: US 2026/0256414 A1
(43) Pub. Date: Sep. 3, 2026

(54) AUTOMATED SEIZURE DETECTION

(71) Applicant: MINDSIGNS HEALTH INC., Mahwah, NJ (US)

(72) Inventors: Justin DAUWELS, Rotterdam (NL); Yuanyuan YAO, Leuven (BE); Wei Yan PEH, Singapore (SG)

(21) Appl. No.: 18/878,864

(22) PCT Filed: Jul. 3, 2023
(86) PCT No.: PCT/US2023/069586

§ 371 Date: Dec. 24, 2024

Related U.S. Application Data
(60) Provisional application No. 63/358,026, filed on Jul. 1, 2022.

(51) Int. Cl. A61B 5/00; A61B 5/37; A61B 5/372

(52) U.S. Cl. CPC A61B 5/4094; A61B 5/37; A61B 5/372; A61B 5/7203; A61B 5/7221; A61B 5/7267

(57) ABSTRACT

A robust seizure detector to detect seizures in a non-patient-specific matter without fine-tuning (if any) is provided. A multi-modular cascading deep and machine learning-based pipeline to detect seizures from channel- to segment- to EEG-level is utilized. For channel-level detection, one of three models is utilized: convolution neural network (CNN), CNN with belief matching (BM) framework (CNN+BM), and Transformer-CNN with BM (CNN+Transformer+BM). Regional features are extracted from the channel-level detector output and a machine learning module for the segment-level detector is applied. For the EEG-level detector, segment-level output is utilized and a convolutional-based postprocessing module is used for seizure detection.

### CROSS-REFERENCE TO RELATED APPLICATIONS

This application claims priority to U.S. Provisional Patent Application No. 65/358,026, filed on Jul. 1, 2022, the disclosure of which is incorporated herein by reference in its entirety for all purposes.

### BACKGROUND OF THE INVENTION

Epilepsy is a brain disorder characterized by the manifestations of abrupt synchronous discharges in great ensembles of neurons in brain structures known as seizures [32]. Individuals diagnosed with epilepsy experience epileptic discharges, which are the paroxysmal activities that arise during seizures (ictal) or between seizures events (interictal) [21]. At any given time, approximately 1% of the world's population are diagnosed with epilepsy [48]. In addition, approximately 10% of the world population will experience an episode of seizure within their lifetime [22]. Overall, provoked and unprovoked seizures occur in about 3.5 per 10000 and 4.2 per 10000 individuals annually [48]. After a seizure episode, the likelihood of encountering another seizure event increases sharply to nearly 50%, bringing the individual to a much greater risk of relapsing [9]. Automating seizure detection in EEG had always been a complicated task. Thus far, the golden standard for seizure detection is still via visual inspection from neurologist [7]. Many attempted to develop an automated seizure detection system to aid neurologists in annotating seizures, but only few models are readily prepared for clinical deployment. Additionally, many existing models are patient-specific, hence not generalizable for different patients. For those non-patient-specific models, the majority of them achieved poor results when validated on large datasets, making them unacceptable for clinical deployment.

Seizure episodes can be visualized and recorded with an electroencephalogram (EEG), which is a method to measure the brain electrical activity across the scalp with surface electrodes [48]. Additionally, one can measure the electrical signals directly from the brain with intracranial EEG (iEEG), where the electrodes are placed directly or implanted onto the brain [43]. Epileptic seizures may occur in the brain locally (partial seizures) or involve the entire brain region (generalized seizures) [42]. The behavior and emotions of a patient can be altered spontaneously and recurrently by seizures events [32]. Moreover, a seizure occurrence may lead to notable outward effects such as uncontrolled shaking and the loss of consciousness or awareness [15].

Clinical neurologists examine short EEG recordings (usually 30 minutes) of interictal periods daily to identify possible neurological abnormalities [30]. The most common forms of interictal events are isolated spikes, sharp waves, and spike-and-wave complexes [22]. These events are perceived in the bulk of patients diagnosed with epilepsy. For this reason, interictal event detection plays a vital role in diagnosing epilepsy. However, those events are not considered clinical seizures. An ictal event consists of different morphology, not limited to rhythmical waveforms for a wide variety of frequencies, polyspike activity, low-amplitude desynchronization, as well as spike-and-wave complexes [42]. Thus, while interictal findings suggest proof of epilepsy, diagnosis of epilepsy is still based on detected epileptic seizures [32].

The interictal indications of epilepsy can be identified with short periods of EEG recording. However, one may deploy long-term video EEG (vEEG) for monitoring due to the infrequent nature of seizure occurrence. Traditionally, epileptic seizures onset is notified or detected either by the subject activating an alarm or by direct observation of outward effects [15]. In addition, the advancement of ambulatory EEG permitted the characterization of epileptic seizures and seizure-like events at any location, such as at home [71].

Overall, visual seizure detection has not been proven cost and time-efficient [25]. Efficient automated seizure detection schemes can facilitate the bottleneck of the diagnosis of epilepsy, enhancing the management of long-term EEG recordings. Unfortunately, automating seizure detection in EEG is complicated. Despite a vast variability in seizure patterns across different patients, seizures are often patient-specific. As such, a patient will typically experience the same type and class of seizure periodically and recurrently. Hence, there are incentives for the seizure detection model to be developed in a patient-specific manner. Thus far, many existing seizure detection models that achieved excellent performance are patient-specific.

However, for generalization purposes, a non-patient-specific approach should be more appropriate. For a seizure detector to be robust and easily deployable, it should be able to detect seizures in any patient. Unfortunately, for non-patient-specific models, the majority achieved poor results when validated on large datasets, which is unacceptable for clinical deployment.

Reviewing the current literature, the majority of the studies validated their seizure detectors on two public seizure datasets, namely the Temple University Hospital seizure (TUH-SZ) dataset [27, 55, 4, 66, 26, 52, 31, 68, 5, 20, 39, 2, 17, 53] and the Children's Hospital Boston Massachusetts Institute of Technology (CHB-MIT) dataset [23, 64, 41, 28, 5]. Among these studies, many deployed different variations of convolutional neural network (CNN) [23, 64, 55, 26, 28, 53, 3, 44, 37], recurrent neural network (RNN) [55, 17], transformers [36, 10, 50], gating algorithm [7], hidden Markov models (HMM) [6], temporal graph convolutional network (TGCN) [19], and logistic regression (LR) [67]. Across different works, the seizure detector deployed are relatively identical in the pipeline: detect seizures at segment-level before advancing to EEG-level seizure detection. Another common trend is that most studies focused on building deeper and more complex neural networks with million to billion neurons and hyperparameters to detect seizures, instead of changing the pipeline [10, 19]. However, even with such computationally expensive models, most detectors failed to enhance non-patient-specific seizure detection results. Furthermore, for those systems verified with bigger private seizure datasets, the results reported were similar to those achieved in earlier works, with minor improvements, if any [19]. There is a bottleneck that limits the performance of non-patient-specific seizure detection in the field. In this work, we attempt to develop a non-patient-specific seizure detector to resolve the bottleneck that accommodates the existing weakness in existing seizure detection models. To do so, we first identify the existing weaknesses and address them accordingly.

Firstly, many existing studies deployed a patient-specific seizure detection approach [11, 34, 54, 10], which is challenging to implement in clinical settings. For a model to be patient-specific, retraining is required prior to seizure monitoring; a sufficient amount of annotated EEG data from the same patient is needed. Such circumstances are rare and difficult to replicate. A non-patient-specific seizure detector resolves this limitation, as the necessity for retraining is eliminated. However, most existing non-patient-specific detectors failed to achieve acceptable results, as seizure morphologies are often patient-specific [17, 55, 27].

A review of published works showcased an apparent similarity of most automated seizure detector pipelines. The standard pipeline begins by detecting seizures in a multi-channel segment (segment-level) with a machine or deep learning model, before detecting seizures in the entire EEG (EEG-level). The main novelty of those works lies solely in the segment-level seizure detection model, where the algorithms differ. The underlying flaw of this approach is that the number of channels must be fixed. Many existing studies fixed the number of channels and deployed 2D deep learning models that require a fixed number of channels as input. By contrast, the present invention first analyzes each channel individually, and then computes a variety of statistics across all channels in subsequent stages (segment-level and EEG-level analysis). As a result, the proposed system can be applied to an arbitrary number of channels. As a result, it is impossible to deploy the same seizure detector on different datasets with a different number of electrodes. Despite this being the most widely used pipeline for automated seizure detection, there appears a fatal weakness within this approach; most work does not account for EEGs with different numbers of EEG channels.

By providing a non-patient-specific seizure detector, the present invention solves these and other shortcomings of prior art seizure detection methods.

### BRIEF SUMMARY OF THE INVENTION

The present inventions addresses many of the issues mentioned above by providing a robust, non-patient specific method of seizure detection. In various embodiments, the method initiates seizure detection at the channel level. The method relies on machine learning models that contain many fewer parameters compared to earlier models that do not detect seizures at individual channels. Therefore, in various embodiments, the method is less prone to overfitting to particular datasets recorded by specific EEG machines at specific institutions, and can achieve stable and consistent results across EEG datasets recorded by a variety of EEG machines at a variety of institutions. Moreover, in exemplary embodiments, the method can be applied to both scalp and intracranial EEG and pediatric and adult EEG, without the need for retraining.

In an exemplary embodiment, the invention deploys a multi-modular cascading deep and machine learning approach model to perform generalized, non-patient specific seizure detection in EEG. In various embodiments, seizures are detected on two or more different EEG scales: e.g., channel-, segment-, and EEG-level. Prior to the current invention, no existing work initiated seizure detection from channel-level to segment- and EEG-level. Beginning with a channel-level detector provides a complete generalization of seizure detection across EEGs with a different number of channels, as long as there is one channel. This enables much easier cross-institutional verification of the seizure detector.

Another utility identified is that while seizure detection can be done on both EEG and intracranial EEG (iEEG), most work only performs seizure detection on a single EEG type. In an exemplary embodiment, the present invention provides a model trained with adult EEG data, which is employed for EEG/iEEG data for neonatal/pediatric/adult patients without the need for retraining in exemplary embodiments.

An exemplary embodiment of the method begins with channel-level and ends with EEG-level seizure detection. For each scale, different approaches are deployed in detecting seizures. For channel-level detection, in various embodiments, three variations of CNN are deployed: CNN, CNN with belief matching loss function (CNN+BM), and CNN with transformer and belief matching loss function (CNN+Transformer+BM). In various embodiments, a Bayesian approach is applied via a belief matching framework and leads to a better generalized seizure detection. An exemplary transformer provides correlation in adjacent data to fine the features extracted by CNN. In various embodiments, for segment-level, predictions were utilized from the channel-level output and further extracted statistical features and distribution and performed classification using machine learning models via those features. In one embodiment, for EEG-level seizure detection, several postprocessing steps such as linear/non-linear filters, simple thresholding, and clustering are applied.

In an exemplary embodiment, the method of the invention is utilized to detect artifacts in EEG.

The present invention provides significant improvements compared to current methods of seizure detection. Objects, advantages and embodiments of the invention are further discussed in the detailed description that follows.

### BRIEF DESCRIPTION OF THE DRAWINGS

FIG. 1. Precision-Recall curve of the channel, segment, and EEG-level seizure classification for all window length across the datasets.

FIG. 2. (a) Stacking histograms of TP and FN were sorted by seizure duration, (b) distribution of SEN per EEG, (c) distribution of PRE per EEG, and (d) distribution of FPR/h per EEG for the different datasets.

FIG. 3. Exemplary seizure detection system. The system consists of multiple modules: a channel-level classifier, a segment-level classifier, a simple thresholding module, and multiple postprocessing filters and modules:
Channel-level detection, where individual EEG channels are processed in short windows of time (typically 5 s to 30 s) in order to determine whether they contain seizure activity or not;
Segment-level detection, where all EEG channels are processed together in short windows of time (typically 5 s to 30 s) in order to determine whether they contain seizure activity or not;
EEG-level detection, where the start and end times of seizures are determined in the entire EEG, in addition to the channels that contain seizure activity.

FIG. 4. The modules in the transformers are illustrated here. A. Scaled Dot-product Attention⁵⁹. B. Multi-Head Attention which consists of several attention layers running in parallel.

FIG. 5. The structure of the modified transformer encoder deployed in this work. A. Modified transformer encoder. B. Modified transformer encoder Incorporated with CNN.

FIG. 6. Table 1. Channel-level seizure detection results for different CNN models.

FIG. 7. Table 2. Table 2 Segment-level seizure detection results for different CNN models.

FIG. 8. Table 3 EEG-level seizure detection results (CNN-Trans-BM model evaluated with MOES, Overlap, and TAES).

FIG. 9. Table 4 EEG-level seizure detection results (evaluated with MOES).

FIG. 10. Precision-recall (PR) curve for the NeuroBrowser detector on the set of 145 EEGs with Persyst as benchmark. FIG. 5A, 5B, 5C, 5D show the results for All, Continuous, Extended, and Routine EEGs respectively.

FIG. 11. Table 5: Detection metrics for different types of NNI EEGs with NeuroBrowser and Persyst. Lat: latency, Ovlp: channel overlap.

FIG. 12. Histograms of detection metrics for NeuroBrowser and Persyst on 145 test EEGs. FIG. 12A1, 12B1, 12C1, 12D1, 12E1, 12F1, 12G1 show results of Neurobrowser for sensitivity, precision, FDR, Lat, Ovlp, sensitivity global, and sensitivity focal respectively. FIG. 12A2, 12B2, 12C2, 12D2, 12E2, 12F2, 12G2 show results of Persyst for sensitivity, precision, FDR, Lat, Ovlp, sensitivity global, and sensitivity focal respectively.

FIG. 13. Precision-recall (PR) scatter and density plots for different types of EEGs with NeuroBrowser and Persyst. Each dot represents an EEG among the 145 test EEGs and the shades represent the distribution of the EEGs on the axes. Darker shades denote a higher density of EEGs at those regions. The limits of all the axes are the same and as indicated in the plot at row two, column three. FIG. 13A1, 13B1, 13C1, 13D1 show the results of Neurobrowser for All, Continuous, Extended, and Routine EEGs respectively. FIG. 13A2, 13B2, 13C2, 13D2 show the results of Persyst for All, Continuous, Extended, and Routine EEGs respectively.

FIG. 14. Table 8: Detection metrics for different types of NNI EEGs with NeuroBrowser and Persyst at different postprocessing thresholds ThE. Lat: latency, Ovlp: channel overlap.

### DETAILED DESCRIPTION OF THE INVENTION

### I. Introduction

In various embodiments, the present invention provides a method of seizure detection via a cascading method (channel-, segment-, and EEG-level) on six datasets via a non-patient-specific approach. We deployed three different models for the channel-level detector (CNN, CNN with BM loss, and transformer with BM loss), a machine learning model for the segment-level detector, and several postprocessing modules for the EEG-level detector. The seizure information is passed on to the posterior module during the training of each cascading module. We trained and evaluated the seizure detector on the TUH-SZ dataset and deployed the trained model to evaluate seizures on five other datasets (CHB-MIT dataset, SWEC-ETHZ dataset, HUH dataset, iEEGP dataset, and EIM dataset). We compared our results against relevant literature and found that our method obtained significantly better results.

The significant improvement can be attributed to certain characteristics of the present invention. In an exemplary embodiment, the method utilizes channel-level seizure detection, which makes seizure detection more precise during training. Furthermore, as the TUH-SZ dataset contains channel-level information, an exemplary embodiment rejects noise and non-seizure channels in seizure-affected segments during training. In some embodiments, using a transformer with BM loss, the channel-level classifier can generalize seizure detection much better than a traditional CNN with an SM loss function.

In one embodiment, following the channel-level detector, the segment-level detector can deploy the same pipeline on EEGs/iEEGs with different electrodes via regional statistics. No prior work has attempted to incorporate a channel-level seizure detector with a segment-level detector to enable seizure detection on any EEGs/iEEGs with any number of channels. The present invention demonstrates, in various embodiments, that a channel-level detector provides an unexpected improvement in seizure detection offsetting EEG low SNR and the noisy nature of EEG data.

Finally, the novelty of the present invention is validated by the fact that previous studies have not reported the seizure evaluation metric deployed in the present invention. Studies have routinely utilized a metric that is not robustly useful, such as the EBS (essentially a segment-level classification).

Reference will now be made in detail to implementation of exemplary embodiments of the present disclosure as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Many modifications and variations of the exemplary embodiments set forth in this disclosure can be made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

Unless defined otherwise, all technical and scientific terms used herein have the same meaning as commonly understood by one of ordinary skill in the art.

### II. Terms

### A. Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutical formulation, and medical imaging are those well-known and commonly employed in the art.

The articles “a” and “an” are used herein to refer to one or to more than one (i.e. to at least one) of the grammatical object of the article. By way of example, “an element” means one element or more than one element. Contrast agents with iodine, barium or other atoms with Z greater than 52 are exemplary “high Z” materials.

A “disease” is a state of health of an animal wherein the animal cannot maintain homeostasis, and wherein if the disease is not ameliorated then the animal's health continues to deteriorate.

“Seizure”, as this term is used herein refers to manifestations of abrupt synchronous discharges in great ensembles of neurons in brain structures in epilepsy patients [31].

“Channel” as this term is used herein refers to the signal recorded by an EEG electrode. “Channel-level” refers to the analysis of brief signals at specific EEG channels [ . . . ], where signals at each channel are analyzed separately.

“Segment” as this term is used herein refers to a multi-channel EEG signal in a brief time period, typically 5 s to 30 s. Segment-level refers to the analysis of the EEG signals from all channels within a brief time period (time segment), typically 5 s to 30 s. In contrast to channel-level analysis, signals from all channels are analyzed collectively instead of separately.

“EEG”, as used herein, refers to electroencephalogram, the recording of electrical signals by electrodes placed on the scalp. “EEG-level refers to the analysis of the entire EEG signal, from all channels and the entire duration.

As used interchangeably herein, the term “classifier” or “model” refers to a machine learning model or algorithm, and vice versa.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neuralnetworks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xᵢ, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in accordance with the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the classifier is a Naive Bayes algorithm. Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍₀=∥x₍₀−x_{(Q)}∥ Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model andHidden Markov model. In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where. n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in an k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

An exemplary algorithm contains more than 1×10⁶ parameters, mainly the filter coefficients and weights inside the CNN, as a consequence, it cannot be executed by mental calculations.

### B. Abbreviations

TP: true positive; FN: false negative; PRE: precision; CNN: convolutional neural network; BM: belief matching; SNR: signal to noise ratio; EBS: Epoch-Based Sampling; OVLP: any-overlap; TAES: Time-Aligned Event Scoring; MOES: Minimum Overlap Event Scoring

### III. Embodiments

In various embodiments, the outputs of the algorithm are displayed on a graphical user interface, together with the EEG traces. The outputs are also stored and listed in a queriable database, that can be used to inspect specific types of detected waveforms, e.g., seizures.

### A. Methods

In various embodiments, the invention provides methods of automated seizure detection. Seizures are deterministic biomarkers of epilepsy. To detect seizures rapidly, reliable automated detection of seizures from scalp electroencephalogram (EEG) is utilized.

In various embodiments, the present invention provides a method employing a transformer with a convolutional neural network (CNN) and belief matching (BM) loss to detect seizures in different EEG scales (channel-, segment-, and EEG-level). First, the transformer is employed to identify seizures at single-channel segments (channel-level). Next, regional features are extracted to predict the seizure probability in multi-channel segments (segment-level). Thirdly, several convolutional-based postprocessing modules on successive segment-level outputs to formulate the seizure detection at EEG-level are applied.

In some embodiments, seizure detection is optionally evaluated in two ways across multiple datasets: First, a 4-fold CV on the Temple University Hospital seizure (TUH SZ) dataset, and then using the system trained exclusively on the TUH dataset on datasets from five other centers: Children's Hospital Boston Massachusetts Institute of Technology (CHB-MIT) dataset, Sleep-Wake-Epilepsy-Center at ETH Zurich (SWEC-ETHZ) dataset, Helsinki University Hospital (HUH) dataset, International Epilepsy Electrophysiology Portal (iEEGP) dataset, and Epilepsy-iEEG-Multicenter (EIM) dataset. Furthermore, we propose the minimum overlap evaluation scoring (MOES) to account for the minimum overlap duration required for a detection to be accurate as our performance evaluation metric. The proposed non-patient-specific seizure detector achieved an average performance of sensitivity (SEN) between 0.644 to 0.765, precision (PRE) between 0.635 to 0.731, false positive rate per hour (FPR/h) between 0.329 to 1.165, and detection offset between −7.954 to 2.180 across the six centers. Deployment of the inventive method in a clinical setting, can significantly improve patient care via enhancing efficiency and precision.

In various embodiments, there is provided an automated method of seizure detection using a method selected from scalp electroencephalogram (EEG), intracranial EEG (iEEG) and a combination thereof, the method comprising:
(a) applying a transformer with a convolutional neural network (CNN) and a belief matching (BM) loss to acquire subject data from at least one single-channel segment (channel-level), acquiring channel-level data;
(b) extracting from the channel-level data at least two regional features, thereby acquiring data from multi-channel segments (segment level);
(c) from the multi-channel segment data, predicting seizure probability in the multi-channel segments;
(d) repeating (c) at least once, acquiring successive segment-level outputs; and
(e) applying at least one convolutional-based postprocessing module on the successive segment-level outputs, thereby detecting the seizure.

In various embodiments, there is provided a method, wherein the transformer is a deep learning model based on attention mechanism.

In various embodiments, there is provided a method, wherein the attention mechanism is self-attention.

In various embodiments, there is provided a method, wherein loss of order information at the channel-level is mitigated by applying positional encoding to channel-level input data.

In various embodiments, there is provided a method, wherein subject data are acquired from at least 10 channel segments, at least 15 channel segments, or at least 20 channel segments.

In various embodiments, there is provided a method, wherein subject data are acquired from at least 21 channel segments.

In various embodiments, there is provided a method, wherein the channel-level data are data from an electroencephalogram.

In various embodiments, there is provided a method, wherein the channel-level data yields seizure probabilities for each channel.

In various embodiments, there is provided a method, wherein the seizure probabilities are arranged into regions according to the scalp topology: frontal, central, occipital, and parietal. Besides those four local regions, we also define a “global” region containing.

In various embodiments, there is provided a method, wherein the seizure probabilities are further arranged into a “global” region containing all channels.

In various embodiments, there is provided a method, further comprising extracting from each region at least one of seven statistical features: mean, median, standard deviation, maximum value, minimum value, and value at 25% and 75% percentile, thereby forming a feature set.

In various embodiments, there is provided a method, wherein there are five regions and 5×7=35 features are extracted.

In various embodiments, there is provided a method, further comprising computing from all channel-level outputs normalized histogram features (5 bins, range [0,1]) and including them into the feature set, bringing the total features in the feature set to 40.

In various embodiments, there is provided a method, wherein the repeating in (d) is performed for all EEG segments.

In various embodiments, there is provided a method, wherein (e) comprises: applying at least one 1D smoothing filter, thereby removing from the data isolated seizure detections (e.g., false positives), and smoothing regions with significant confidence variations, and stabilizing the detections.

In various embodiments, there is provided a method, wherein (e) further comprises: following smoothing, thresholding to the seizure probabilities to round them to zeros (seizure-free) or ones (seizure).

In various embodiments, there is provided a method, wherein a threshold value θ∈{0.1, 0.2, 0.3, 0.4. 0.5, 0.6. 07, 0.8, or 0.9} is utilized.

In various embodiments, there is provided a method, further comprising: following thresholding, identifying consecutive is of length smaller than Nc, and replacing the identified 1 s with 0 s, thereby removing short detections, leading to fewer false positives and more false negatives, as the system may miss short seizures.

In various embodiments, there is provided a method, wherein Nc∈{1, 2, 3, 4, 5, 6, 7, 8. 9. 20, 11, 12, 13, 14, 15, 16, 17, 18, 19, or 20}.

In various embodiments, there is provided a method, wherein following the replacing remaining sequences of consecutive is are identified, and the start and end time of the consecutive is is identified, thereby resulting in a final output of the EEG-level seizure detector being the start and end times of the detected seizures.

The following Examples are offered to illustrate exemplary embodiments of the invention and do not define or limit its scope.

### EXAMPLES

### Example 1

### 1.1 Materials and Methods

Firstly, we deployed a multi-modular cascading deep and machine learning approach model to perform generalized seizure detection in EEG. We detect seizures on different EEG scales: channel-, segment-, and EEG-level. We begin with channel-level and end with EEG-level seizure detection. For each scale, deployed different approaches in detecting seizures. For channel-level detection, we deployed three variations of CNN in this study: CNN, CNN with belief matching loss function (CNN+BM), and CNN with transformer and belief matching loss function (CNN+Transformer+BM). It is believed that a Bayesian approach via a belief matching framework can better generalize seizure detection. Moreover, with a transformer, one can find correlation in adjacent data to fine the features extracted by CNN. Next, for segment-level, we utilized the predictions from the channel-level output and further extracted statistical features and distribution and performed classification using machine learning models via those features. Finally, for EEG-level seizure detection, we applied several postprocessing steps such as linear/non-linear filters, simple thresholding, and clustering.

Secondly, we evaluated the seizure detector on six different datasets with different EEG types and patient age: Temple University Hospital Seizure (TUH-SZ) dataset, Children's Hospital Boston Massachusetts Institute of Technology (CHB-MIT) dataset, Sleep-Wake-Epilepsy-Center at ETH Zurich (SWEC-ETHZ) dataset, Helsinki University Hospital (HUH) Neonatal EEG dataset, International Epilepsy Electrophysiology Portal (iEEGP) iEEG dataset, and Epilepsy-iEEG-Multicenter (EIM) dataset. Moreover, we aim to perform transfer learning to verify if it is possible to train the seizure detector on one EEG type or patient age group and deploy it on another EEG type and patient age group. As such, we trained the seizure detection system with data from the TUH SZ dataset and evaluated it on the five other datasets. Therefore, we select the TUH SZ dataset as the primary training dataset, the largest public dataset available. Additionally, it contains channel-level seizure annotations, which allow better channel-level seizure detection training. Finally, every seizure is annotated with its seizure type (eight seizure types), showcasing a massive variety of seizure classes within the dataset.

Thirdly, we only deploy patient-specific models for evaluation. Hence, all the evaluations are performed by splitting patients into different folds if the dataset contains patients with multiple files. This ensures that the system does not learn from data from the same patient, which may over-fit the seizure detection system. Lastly, we defined an objective evaluation metric for seizure detection known as the Minimum Overlap Event Scoring (MOES) for a more effective seizure detection measurement. The metric allows us to define what constitutes a true detection, which is critical for EEG-level seizure detection.

With the improvements and focus as compared to the current seizure detection literature, this paper hopes to improve the seizure detection performance for non-patient-specific seizure detection.

### 1.2 Methods

### Dataset

In this work, we employ six public datasets to validate our seizure detection model:
1. Temple University Hospital Seizure (TUH-SZ) dataset⁴¹.
   https://isip.piconepress.com/projects/tuh_eeg
2. Children's Hospital Boston Massachusetts Institute of Technology (CHB-MIT) dataset⁴⁰.
   https://physionet.org/content/chbmit/1.0.0/
3. Sleep-Wake-Epilepsy-Center of the University Department of Neurology at the Inselspital Bern at ETH Zurich (SWEC-ETHZ) dataset^{4x}.
   http://ieeg-swez.ethz.ch/
4. Helsinki University Hospital (HUH) dataset)⁴⁷.
   https://zendodo.org/record/2547147
5. International Epilepsy Electrophysiology Portal (iEEGP) dataset by the University of Pennsylvania and the Mayo Clinic.
   https://www.ieeg.org/
6. Epilepsy-iEEG-Multicenter (EIM) dataset⁴⁴.
   https://openneuro.org/datasets-ds003029/versions/1.0.3

The detailed information of the EEG/iEEGs of the six datasets are displayed in Table 6.

TABLE 6
Information on the six datasets deployed in the study

| General | Description | TUH-SZ | CHB-MIT | SWEC-ETHZ | HUH | iEEGP | EIM |
|---|---|---|---|---|---|---|---|
| EEG/iEEG | Patient Type | Human | Human | Human | Human | Human/Dog | Human |
| Information | Patient Age | Adult | Pediatric | Adult | Neonatal | Adult | Adult |
| | EEG Type | EEG | EEG | iEEG | EEG | iEEG | EEG/iEEG |
| | Sample | 250, 256, 400, 512 | 256 | 512 | 256 | 400, 500 | 250, 500 |
| | Frequency (Hz) | 1000 | | | | 5000 | 1000 |
| | Channel name | Available | Available | Unavailable | Available | Available | Available |
| | SZ Channel | Yes | No | No | No | No | No |
| | SZ Type | Yes, 8 | No | No | No | No | No |
| | No of Channels | 19, 21 | 23, 24, 26 | 36-100 | 21 | 16-72 | 53-216 |
| Dataset | No of Patients | 637 | 24 | 16 | 75 | 12 | 31 |
| Information | All EEG | 5610 | 683 | 100 | 75 | 12 | 102 |
| | Non-SZ EEG | 4457 | 545 | 0 | 22 | 0 | 0 |
| | SZ EEG | 1153 | 138 | 100 | 54 | 12 | 102 |
| | SZ Events | 3055 | 185 | 100 | 517 | 12 | 102 |
| Duration | All EEG | 922.673 | 979.929 | 13.521 | 78.55 | 7.201 | 7.957 |
| (hr) | Non-SZ EEG | 680.659 | 792.419 | 0 | 34.961 | 0 | 0 |
| | SZ EEG | 242.015 | 187.51 | 13.521 | 78.55 | 7.201 | 7.957 |
| | Average (All) | 0.164 | 1.435 | 0.135 | 1.494 | 0.600 | 0.078 |

The TUH-SZ dataset is a large dataset suitable for large-scale EEG seizure detection⁴⁵. The seizures in this dataset are annotated based on seizure type and location (channel-based), allowing filtering of non-seizure-affected channels for training the channel-level seizure detector.

The CHB-MIT dataset consists of EEG recordings collected from 23 pediatric subjects from 24 cases (5 males, ages 3-22, 17 females, ages 1.5-19, and 1 unknown with no age information)⁴⁶. Each patient has multiple seizures and non-seizure EEGs assigned to them, with each EEG having a duration of at least an hour. Many works had performed seizure detection using a patient-specific approach, with few tackling it in a non-patient-specific approach^{48,49}.

The SWEC-ETHZ database is a short-term iEEG dataset, consisting of 100 seizures from 16 patients³⁸. The patients are evaluated for epilepsy surgery. However, the channel names and locations are not available in this dataset. All the iEEG files contain one seizure with a minimum duration of at least 10 s, with 3 minutes of preictal and postictal segments at the start of every seizure. The channel names of the EEGs are not provided for this dataset.

The HUH EEG dataset is a neonatal seizures dataset recorded from the neonatal intensive care unit (NICU) and visually interpreted by the human expert. This dataset comes with three sets of seizure annotation by three experts. To finalize the final annotation, we compared the annotation across the annotators and establish the final annotation via hard voting. As a result, only 22 EEGs are completely seizure-free by consensus, while the remaining have seizure annotated by at least one annotator.

The iEEGP dataset was created by the University of Pennsylvania and the Mayo Clinic initially for a seizure detection challenge on kaggle.com. Later, the iEEG portal was developed to create the most extensive public iEEG dataset for public annotation.

The EIM dataset contains both iEEG and EEG data from four centers with a total of 103 subjects. The dataset contains data from four centers: University of Maryland Medical Center (UMMC), University of Miami Jackson Memorial Hospital (UMH), National Institute of Health (NIH), and Johns Hopkins Hospital (JHH). All the EEGs and iEEGs in this dataset contain exactly one seizure each.

Ultimately, the TUH-SZ dataset is the largest across all six datasets in terms of unique patients, seizures duration, and events. Hence, we feel that the TUH-SZ dataset is the most appropriate as the primary data source for the training of the seizure detection model. Then, we intend to deploy the model pretrained with data from the TUH-SZ dataset to the other datasets to verify if the approach works even for different datasets:
1. EEG type: EEG/iEEG
2. EEG recording device
3. Patient age group: neonatal/pediatric/adult
4. Patient type: Human/dog

### 1.2b EEG Preprocessing

We apply several preprocessing steps on the EEGs: a Butterworth notch filter (4^{th} order) at 60 Hz (USA) to remove the electrical interference of power signals, a 1 Hz high-pass filter (4^{th} order) to remove the DC shifts and baseline fluctuations. Next, we standardized the gain of all the EEG files and downsampled all the EEGs to 128 Hz. Finally, for EEGs with channel names provided, we convert all the EEG signals from monopolar to bipolar montages, as all the seizures are annotated in the bipolar montage.

### 1.2c EEG Seizure Detection Pipeline

We perform the seizure detection on channel-, segment-, and EEG-level, in that order. Such cascading approach enables optimization within individual modules via a systematic approach. The pipeline is similar to the structure to the EEG classifiers described in⁵⁰⁻⁵⁷. We illustrate the seizure detection system in FIG. 3.

### 1.2d Channel-Level Binary Seizure Detection

Across multiple works, the window lengths W deployed for seizure detection lies between 1 s to 30 s. However, 1 s is too short of capturing a seizure morphology, and is more suitable for detecting Interictal Epileptiform Discharges (IEDs), while 30 s is too long to capture shorter seizure episodes. Hence, we varied the single-channel segment duration window from 3, 5, 10, and 20 s to determine the appropriate window length to capture the morphology and features of a seizure. The channel-level seizure detector identify seizure and non-seizure single-channel EEG signals segments (see FIG. 3).

### 1.2e Convolutional Neural Network (CNN)

We deploy a Convolutional Neural Network (CNN), where the input is the raw EEG single-channel signal. The input shape has a dimension of W×128. We implemented the CNN in Keras 2.2.0 and TensorFlow 2.6.0⁵³. The details of the CNN model are described in Table 7. The optimizer used in the system is Adam with an initial learning rate equals to 10⁻⁴, which minimizes the cross-entropy. The batch size is 1000. To avoid overfitting during training, we applied balanced training by implementing weights corresponding to the inverse of each class's number of each sample. Finally, we optimized hyperparameters within the CNN with a nested CV on the training data with an 80.20% split for training and validation⁵⁰.

TABLE 7
CNN model.

| | Number | | Output | Activation |
|---|---|---|---|---|
| Layer type | of filters | Filter size/stride | shape | function |
| Input | 1 | −/− | 1 | − |
| Convolution 1D | 8 | 1 | 8 | ReLU |
| Max Pooling ID | − | 1 | 8 | − |
| Convolution 1D | 16 | 1 | 16 | ReLU |
| Max Pooling ID | − | 1 | 16 | − |
| Convolution 1D | 32 | 1 | 32 | ReLU |
| Max Pooling ID | − | 1 | 32 | − |
| Convolution 1D | 64 | $( W * Fs ) \times ( W * Fs - 2 ) \times ( \frac{1}{2} ( W * Fs ) - 1 ) \times ( \frac{1}{2} ( W * Fs ) - 3 ) \times ( \frac{1}{4} ( W * Fs ) - \frac{3}{2} ) \times ( \frac{1}{4} ( W * Fs ) - \frac{7}{2} ) \times ( \frac{1}{8} ( W * Fs ) - \frac{7}{4} ) \times ( \frac{1}{8} ( W * Fs ) - \frac{15}{4} ) \times ( \frac{1}{16} ( W * Fs ) - \frac{15}{8} ) \times ( \frac{1}{16} ( W * Fs ) - \frac{31}{8} ) \times ( \frac{1}{32} ( W * Fs ) - \frac{31}{16} ) \times ( \frac{1}{32} ( W * Fs ) - \frac{31}{16} ) * 128$ | 64 | ReLU |
| Max Pooling ID | − | 1 | 64 | − |
| Convolution 1D | 128 | 1 | 128 | ReLU |
| Max Pooling ID | − | 1 | 125 | − |
| Flatten | − | −/− | | − |
| Fully Connected | − | −/− | 100 | − |
| Dropout | − | −/− | 100 | ReLU |
| Fully Connected | − | −/− | 2 | Softmax |

W is the window length (3, 5, 10, and 20s), and Fs is the sampling frequency at 128 Hz.

### 1.2f Belief Matching Framework

For the reliability in an actual application, it is not sufficient to construct a system with high accuracy. Knowing when the prediction is likely to be incorrect is also essential, such knowledge enabling users to reject specific outputs, apply more delicate operations, or ask real experts to be involved. Therefore, a good uncertainty estimation is required. An intuitive way to acquire the confidence of the predicted label is to interpret the outputs of the softmax (SM) as the categorical probability. To do so, we express x to be the input of a neural network, f^{W} to be the classification model and

$${f}_{k}^{W}$$

to be the k-th output basis of it. Then, the logit is

$${f}_{k}^{W}$$

and the SM output can be written as:

$${ϕ}_{k} ( {f}^{W} ( x ) ) = \frac{exp ( {f}_{k}^{W} ( x ) )}{{\sum }_{j} exp ( {f}_{j}^{W} ( x ) )} , k ∈ \{ 1 , … , K \} \qquad ( 1 )$$

where K is the number of classes. As Σₖ φₖ(f^{W}(x))=1, it is appealing to observe the output of softmax as an approximation of the categorical distribution. However, previous studies have shown that softmax tends to be over-confident and have poorly calibrated behavior⁵⁴⁻⁵⁶. Therefore, while it appears reasonable, in reality it may not a good choice for uncertainty estimation.

To obtain better uncertainty estimation, Joo et al. they proposed a Bayesian approach on the categorical probability: regard it as a random variable instead of a deterministic one. [33]. Then, viewing the binary classification problem from a distribution matching perspective results in the belief matching (BM) loss function. Their experiments have shown that uncertainty estimation and calibrated behavior can be improved by substituting standard deep learning models' classical SM cross-entropy loss with the BM loss. They also observed improvements in generalization performance, the desired property required in our seizure detection system. Therefore, we decided to incorporate the proposed BM framework into our system and see if it can also provide consistent improvements in a different application across other datasets. As our study involves six different datasets, this approach is extremely applicable. For completeness, we will show how to reach the mathematical expression of the proposed BM loss concisely.

Before deriving the BM loss, it is insightful to also interpret the SM loss from the distribution matching perspective.

Given the training samples

$$D = \{ {x}^{( i )} , {y}^{( i )} \} \frac{N}{i = 1} ∈ {( X \times Y )}^{N} ,$$

we can obtain an empirical target distribution. If we regard the outputs of SM to be the parameters of the categorical distribution P^{C}(φₖ(f^{W}(x))), the SM loss can be rewritten (by adding some constant terms) as the KL divergence between the empirical target distribution and the categorical distribution⁵⁷. However, the problem is that the training set is finite and thus does not contain all possible values of x. Also, the estimation of their label frequency can be unreliable for inputs that rarely occur in the training set. Therefore, the empirical target distribution is not a good estimator of the target distribution. Since minimizing the KL divergence, or the SM loss only approximates the empirical target distribution, the model may suffer from severe performance degradation when the inputs of the testing set are significantly different from the training set.

Now, we formally formulate the distribution matching problem under the framework that the categorical probability z is a random variable. Denote the prior of z given the input x as p_{z|x}(z) and the likelihood of z about the label y as p_{z|x}(z)=P^{C}(z|x). As the likelihood is a categorical distribution, it will be easier to assume p_{z|x}(z) to be conjugate prior, i.e., the Dirichlet distribution:

$$P z \vert {x}^{( z )} = \frac{Γ ( {β}_{0} )}{{Π}_{j} Γ ( {β}_{j} )} {\prod }_{k = 1}^{K} {z}_{k}^{{β}_{k} - 1} , {β}_{j} > 0 \qquad ( 2 )$$

$$Where Γ ( \cdot ) is the gamma function , {P}_{i Z i} = 1 and {β}_{0} = {P}_{i β i} .$$

Then the posterior is also a categorical distribution that can be directly obtained given the dataset D using the property of the conjugate family:

Pz|x,y=P^{D}(β+c^{D}(x)) (3)

where β is the stack of concentration parameters βᵢ, and c^{D}(x) is a vector-valued function that counts label frequency. Equation 3 is the target distribution we would like to approximate.

In⁵⁷ the approximate distribution was also modelled as a Dirichlet distribution. More specifically, they assumed the concentration parameter to be the exponential of the logits.

Denoting α^{W}=°f^{W}, then the approximate posterior is:

$$q \frac{W}{z \vert x} ( z ) = \frac{Γ ( {α}_{0}^{W} ( X ) )}{{Π}_{j} Γ ( {α}_{j}^{W} ( x ) )} {\prod }_{k = 1}^{K} {z}_{k}^{Γ ( {α}_{k}^{W} ( x ) ) - 1} \qquad ( 4 )$$

$$where {α}_{0}^{W} ( x ) = {\sum }_{i} {α}_{i}^{W} ( x ) .$$

To approximate Equation 4 to 3, we need to minimize the KL divergence

$$K L ( {q}_{z \vert x}^{W} \vert \vert {P}_{z \vert x , y} ) ,$$

which is equivalent to maximize the evidence lower bound (ELBO)⁵⁸:

$${l}_{EB} ( y , {α}^{W} ( x ) ) = {E}_{{q}_{z \vert x}^{W}} [ log p ( y \vert x , z ) ] - K L ( {q}_{z \vert x}^{W} \vert \vert {P}_{z \vert x , y} ) \qquad ( 5 )$$

After calculations, the analytical form of each term in Equation 5 can be found as follows:

$$E q z W \vert {x}^{[ lo g p ( y \vert x , z ) ] = ψ ( {α}_{y}^{W} ( x ) ) - ψ ( {α}_{0}^{W} ( x ) )} \qquad ( 6 )$$

$$KL ( {q}_{z \vert x}^{W} \vert \vert {P}_{z \vert x , y} ) = log ( \frac{Γ ( {α}_{0}^{W} ( x ) {\prod }_{k} Γ ( {β}_{k} )}{{\prod }_{k} Γ ( {α}_{k}^{W} ( x ) Γ ( {β}_{k} )} ) + {\sum }_{k} ( {α}_{k}^{W} ( x ) - {β}_{k} ) ( ψ ( {α}_{k}^{W} ( x ) ) - ψ ( ( {α}_{0}^{W} ( x ) ) \qquad ( 7 )$$

where ψ(·) is the digamma function. For a mini-batch, the BM loss is defined as:

$$ℒ ( W ) = - \frac{1}{m} {\sum }_{i = 1}^{m} {l}_{EB} ( {y}^{( i )} , {α}^{W} ( {x}^{( i )} ) ) \qquad ( 8 )$$

which can be computed by substituting Equation 5, 6, and 7. In application, we can simply replace the SM loss function layer of the model and with the BM loss function.

### 1.2g Transformer-CNN

The input of the channel-level detector is a long time series sequence. Although a CNN is relatively decent at identifying correlations within adjacent data, it is likely to fail in finding connections between data that are relatively far apart. This inherent limitation makes CNN not very suitable for temporal or sequential data. To compensate for this limitation, we applied a transformer to further refine the feature extracted by CNN.

The transformer is a deep learning model based on attention mechanisms. Given a set of key-value pairs {(k₁, v₁), . . . , (kₘ,vₘ)}, for a new query q, the transformer computes a prediction output. The output is the weighted sum of the values, where the weights are the similarities of the key and query. The measurements of the similar are the attention scoring functions, typically the scaled dot-product. Assuming that the query and the keys have the same length dₖ, and the values are of length dᵥ, the scaled dot-product attention scoring function is:

$$a ( q , {k}_{i} ) = \frac{{q}^{T} {k}_{i}}{\sqrt{{d}_{k}}} \qquad ( 9 )$$

Next, to handle mini-batches, it is more efficient to pack n queries, m keys and values into matrices Q∈ [symbol] , K∈ [symbol] and V∈ [symbol] , respectively. The matrix of the outputs can be written as:

$$h = Attention ( Q , K , V ) = softmax ( \frac{Q {K}^{T}}{\sqrt{{d}_{k}}} ) V \qquad ( 10 )$$

various dependencies (e.g., short-term and long-term), then it is helpful to formulate different representation subspaces. Mathematically, with d_{model}-dimensional queries, keys and values, and h heads:

$$MultiHead ( Q , K , V ) = Concat ( {h}_{1} , ⋯ , {h}_{h} ) {W}^{O} \qquad ( 11 )$$

wherein

$${h}_{i} = Attention ( {QW}_{i}^{Q} , {KW}_{i}^{K} , {VW}_{i}^{V} ) ,$$

such that

$${W}_{i}^{Q} ∈ {ℝ}^{{d}_{model} \times {d}_{k}} , {W}_{i}^{K} ∈ {ℝ}^{{d}_{model} \times {d}_{k}} , and {W}_{i}^{V} ∈ {ℝ}^{{d}_{model} \times {d}_{v}}$$

are parameter matrices of head i and dₖ=dᵥ=d_{model}/h. The visualizations of the scaled dot-product attention and the multi-head attention are shown in FIG. 4.

However, in many applications, there may not be extra information besides the sequential data itself. In such cases, the input is the query, key, and value at the same time, which is called self-attention⁶⁰. Similar to Equation 11, we have.

$$MultiHead ( X ) = Concat ( {h}_{1} , ⋯ , {h}_{h} ) {W}^{O} \qquad ( 12 )$$

$$where {h}_{i} = Attention ( {XW}_{i}^{Q} , {XW}_{i}^{K} , {XW}_{i}^{K} ) .$$

For the channel-level detection system, we applied a modified transformer encoder (see FIG. 5(a)). The multi-head attention module is the same as in FIG. 4 with expression of Equation 12. To avoid losing the order information of the input sequence, positional encoding is applied⁶¹. More specifically, the position information is

For the channel-level detection system, we applied a modified transformer encoder (see FIG. 5(a)). The multi-head attention module is the same as in FIG. 4 with expression of Equation 12. To avoid losing the order information of the input sequence, positional encoding is applied⁶¹. More specifically, the position information is incorporated into the frequencies of sine and cosine functions:

$$P {E}_{( pos , 2 i )} = sin ( pos / {10000}^{( 2 i / {d}_{model} )} ) \qquad ( 13 )$$

$$P {E}_{( pos , 2 i + 1 )} = cos ( pos / {10000}^{( 2 i / {d}_{model} )} ) \qquad ( 14 )$$

where i is the dimension and pos is the position. To reduce training time, layer normalization proposed in⁶² is used. The feed forward module consists of two linear transformations⁵⁹:

$$FNN ( x ) = max ( 0 , {xW}_{1} + {b}_{1} ) {W}_{2} + {b}_{2} \qquad ( 15 )$$

The resulting structure of the modified channel-level detector is shown in FIG. 5(b). Firstly, we divide the W-second single-channel EEG window into even shorter local segments, and feed them all into a CNN. The structure of the CNN module is similar to the one described before in Table 7, but with no flattening operation nor fully connected layers. Instead, a sequence of the CNN features is forwarded to the transformer encoder as in FIG. 5 to refine the CNN features, which are concatenated to form the window features. Finally, the window features are passed through two fully connected layers with 100 neurons and two neurons, respectively, and outputs the final window prediction labels.

1.2 h Incorporating Transformer with CNN and the BM Loss Function

To apply BM loss into the CNN, we must select the value of βᵢ, i∈{1, . . . , K}. β controls the impact of the prior distribution on the posterior distribution. For simplicity, we assume βᵢ=β ∀i. To avoid the posterior being dominated by the prior, a small β should be chosen. However, this leads to small αᵢ^{W}(x) and large gradients of the ELBO, which may cause gradient explosion⁵⁷. To curb this issue, we can set β=1 and introduce a new hyperparameter A, before rewriting Equation 5 as:

$${l}_{EB} ( y , {α}^{W} ( x ) ) = {E}_{{q}_{z \vert x}^{W}} [ log p ( y \vert x , z ) ] - λ K L ( {q}_{z \vert x}^{W} \vert \vert {P}^{D} ( 1 ) ) \qquad ( 16 )$$

and search for λ instead. This transformation does not affect the local optima, and improves the overall stability of the algorithm. In addition, we added a batch normalization layer after the first dense layer and select a small batch size and learning rate (lr). After careful selection, we set the batch size to 32, lr to 10⁻⁴, and λ to 0.1.

To apply the transformer into the CNN, we define the local segment to be is, and the overlap rate to 25%. We set these values for overall performance and the computation efficiency. We fixed the number of heads to 8, and the number of neurons of the hidden layer of the feed forward neural network (FNN) module was 1024. The CNN module attached before the transformer contains the same architecture, with the exception of no flattening operation and fully connected layers.

### 1.2i Segment-Level Seizure Binary Classification: Machine Learning Model

We perform channel-level classification on the channels in a multi-channel segment to obtain a list of probability outputs between [0,1]. With these probabilities, we extract features that applies for EEGs with any number of channels. With knowledge of the EEG channel location and their associated probabilities, we binned the channels into five scalp regions: frontal, central, occipital, parietal, and global (all the channels on the scalp). If the channel information is unavailable, all five scalp regions will be set as global to maintain the same number of regions for consistency during training and testing. Regional-based statistics features allow EEGs with any channels to be inputted, allowing flexibility of any type of EEGs to be classified. Finally, from each regions we extracted standard statistical features (mean, median, standard deviation, maximum, and minimum) and normalized histogram features (5 bins, range set at [0,1]), resulting ins in 5×10=50 features.

Prior to training of the machine learning classifiers, three data feature engineering processing steps are applied to the training data. First, any features with standard deviation of values ≤10⁻⁷ are rejected. Then, the features are normalized to a Normal Distribution N(0,1). Finally, Synthetic Minority Over-sampling Technique (SMOTE) (k=5) is applied to construct synthetic samples for balanced training⁶³. Finally, we employ different machine learning models to determine if the multi-channel segment contains seizure. We applied six learning classifiers in this study: Logistic Regression (LR)⁶⁴, Support Vector Machine (SVM)⁶⁵, Gradient Boosting (GB)⁶⁶, AdaBoost, (AB)⁶⁷, Random Forest (RF)⁶⁸, and XGBoost (XGB)⁶⁹. We determine the best model and hyperparameters with GridSearch Cross-Validation (CV).

### 1.2j Channel- and Segment-Level Seizure Classification Performance Metrics

The performance of the channel- and segment-level classification will be defined with the accuracy (ACC), balanced accuracy (BAC), sensitivity (SEN), specificity (SPE), and F1-score (F1)⁵⁰. As the seizure and non-seizure classes are typically imbalanced, we evaluate the results mainly in BAC.

### 1.2k EEG-Level Seizure Detection: Postprocessing

Finally, we perform EEG-level (multi-channel full EEGs) seizure detection. Here, we aim to detect the seizure start and end time, hence a window onset detection instead of a binary classification. In other words, in addition to detecting a seizure event, we must accurately predict the start and end of the seizure event. To do so, we apply a sliding window of length W with an overlap percentage o to the EEG to extract n number of segment epochs from an EEG with duration T.

$$n = \{ \begin{matrix}1 + ⌊ \frac{T - W}{W ( 1 - o )} ⌋ & if T \geq W \\ 0 & if T < W ,\end{matrix} \qquad ( 17 )$$

The overlap percentage o is set such that the overlap duration is fixed at is. Fixing the overlap duration rather than the overlap percentage standardizes the system for different window lengths and can reduce computation time. Next, we performed segment-level seizure prediction on each epoch to obtain a list of seizure probabilities X=[x₁, x₂, x₃, . . . , xₙ]. This list of seizure probabilities can be considered as a time series of seizure probabilities for an EEG. From the seizure probabilities, we can locate time windows of high seizure concentration. Firstly, we apply a series of postprocessing steps to eject noise from the time series:
1. Apply a 1D linear/non-linear smoothing filter K (mean, median, maximum, minimum, or Gaussian) of different filter lengths K_{f} (3, 5, and 7) with an overlap of 1 sample. The smoothen filter can remove isolated seizure detections, which are usually suspected to be FPs. Additionally, the smoothen filters denoise the region with huge fluctuations due to noise or artifacts, reducing the occurrence of multiple seizures with a short interval between them.
2. Apply simple threshold (Th) to the seizure probabilities [0,1] to round them to 0 s or 1 s. The threshold is varied with a step of 0.1, from 0.1 to 0.9. The outputs revealed the location of seizures, where 0 implies a non-seizure epoch and 1 implies a seizure epoch.

$${x}_{n} = \{ \begin{matrix}1 & if {x}_{n} \geq Th \\ 0 & otherwise .\end{matrix} \qquad ( 18 )$$

3. Apply seizure cluster threshold (Nc) to locate seizure cluster with at least Nc consecutive detections (value of 1). Ne is varied between 1 to 20 to determine the best value. Any seizure clusters with less than Nc consecutive is are discarded to ensure that the system only pings when seizures longer than a certain duration are detected to reduce FPs.
4, Finally, when two or more seizure detections are in proximity, they are combined into a single detection. Combining two close seizures prevents multiple short detections within proximity, resulting in many TPs within the same region. Such a situation will make the results appears better than in actuality. For two consecutive detection interval [t₁,t₂] and [t₃,t₄], we combine the intervals if.

$$[ {t}_{1} , {t}_{2} ] ⊕ [ {t}_{3} , {t}_{4} ] = \{ \begin{matrix}[ {t}_{1} , {t}_{4} ] & if \vert {t}_{3} - {t}_{2} \vert \leq 10. \\ , [ {t}_{3} , {t}_{4} ] & otherwise .\end{matrix} \qquad ( 19 )$$

5. The resulting seizure clusters are the final seizure detection detected by the seizure detection system.

### 1.2l EEG-Level Seizure Detection Evaluation Metric

After the seizure detector detected seizure windows, we examined the outputs to determine if they were correct. The consensus is that the detection must overlap with the actual annotated seizures. However, that is the bare minimum criteria. The amount of overlap required is still disputable. To eliminate ambiguity, one must adequately define the seizure evaluation metric to compute the true positive (TP), false negative (FN), and false positive (FP) of the seizure detector. There are some well-defined metrics, such as the epoch-based sampling (EBS), any-overlap (OVLP), and time-aligned event scoring (TAES) [73]. However, the majority of the papers do not declare the metric they deployed.

Additionally, these metrics may not be defined inadequately to measure the performance of a seizure detector. Of the existing well-defined metrics (EBS, OVLP, and TAES), we felt that these methods do not accurately reflect a seizure detector requirement needed in a clinical setting. Hence, it is necessary to define a new metric that properly reflect the needs of a clinical setting. Before defining the new metric, we justify the need for a new metric by elaborating on the flaws of the existing metrics and how the new metric rectify them.

The EBS metric is suitable for segment-level binary seizure classification. The metric perform binary classification on segments extracted with sliding windows one-to-one. In other words, the metric place more weights in more prolonged seizures, as more epochs can be extracted from them. Meanwhile, shorter seizures that translate to lesser epochs are prioritized less. As a result, the metric measured in EBS no longer reflect the actual seizure detection problem. For instance, for an EEG with a single prolonged seizure, the EBS metric can report multiple TPs, even when there is only a single seizure event. Moreover, as the EBS ignores seizure duration and seizure windows, one will not be able to compute the detection offset. As such, the metric is incapable of yielding information on the seizure duration nor its start and end time.

Next, the OVLP resolves the issue with EBS by checking seizure windows to annotation windows. The OVLP considers that a detection is a TP only if the detection window has a non-zero overlap with the seizure annotation. Hence, the metric focuses on detecting seizure events and does not place greater weights on detecting longer seizures. Unfortunately, the OVLP does not account for the percentage of overlap. This means that if is of a 100 s long seizure is detected, OVLP will report 1 TP even when the system missed 99% of the seizure. The effects are further amplified if the seizure is longer. Additionally, if the detector detects the entire EEG as a seizure, as long as the EEG contains n seizure, it will report n TP, even when all the seizures are encapsulated in a single detection. Otherwise, if there are no seizures, it will just report an FP. Hence, the OVLP can artificially boost the performance of the seizure detector, presenting better results than in reality.

Firstly, TAES deems that if a single detection detects multiple seizures, only the first seizure is considered a TP; the remaining detections are FN. Next, the TAES accounts for the overlap percentage of the detection, and computes TP based on the fraction of the seizure detected, while the remaining fraction of the detection that missed the seizure is an FP. In other words, the TPs, FPs, and FNs are computed in terms of fractions:

$$T P = \frac{DO}{D A} \qquad ( 20 )$$

$$FN = \frac{D A - DO}{D A} = 1 - TP \qquad ( 21 )$$

$$FP = \frac{D D - D}{D D} \qquad ( 22 )$$

where DO is the duration of overlap between detection and annotation, DA is the duration of annotation, and DD is the duration of detection.

While the TAES resolved some issues in OVLP, we identified some problems with its definition. Firstly, if a system detects multiple seizures in a single detection, one should not penalize the system by treating every seizure after the first as FN. As the system had detected the seizures correctly, but failed to consider the gap between successive seizures as background, one should instead penalize the system by treating each gap as an FP. Secondly, having a fraction of TP and FP when a seizure is detected may be highly confusing. For the OVLP metric, if the system reported 1 TP and 1 FP, it can be easily understood that the system had two detections, and one detection is wrong. As such, the precision is 0.5. However, for TAES, 1 TP and 1 FP may imply very different scenarios. For simplicity, we only list down two extreme scenarios. In the first case, the system had two detections, and one detection was perfectly correct, and the other detection was wrong. In the second case, the system had two detections, and both detections detected precisely half of a seizure each. As such, each detection results in 0.5 TP and 0.5 FP. In both scenarios, we will get a precision of 0.5. However, for the second case, catching half the seizure duration might be more than sufficient to determine if a seizure exists.

In other words, the TAES may over-penalize the system. Moreover, reporting fractions of TP or FP to clinicians is not informative and can be confusing. Generally, the TAES report results in a manner that is difficult to comprehend in the real world. Moreover, the metric obtained from the TAES seem to be more applicable for engineering purposes and not for clinical applications.

Hence, we need a new metric that leverage the advantage of the above metrics while eliminating their flaws.

### 1.2m Minimum Overlap Event Scoring (MOES)

The minimum overlap event scoring (MOES) is a seizure evaluation metric that determines a minimum overlap duration before recording a TP or FP. After consulting neurologists for their opinion, it is determined that a seizure annotation should have a minimum duration Tₘᵢₙ of approximately 10 s for it to be pronounced. Next, if two consecutive seizures events are in proximity and are less than Tₘᵢₙ=10 s apart, they should be interpreted as a single event. Hence, a TP is recorded when the overlap duration between the detection (T_{detection}= [dₛₜₐᵣₜ, d_{end}]) and the event (T_{seizure}=[sₛₜₐᵣₜ, s_{end}]) is at least Tₘᵢₙ=10 s, regardless of the seizure event duration. Otherwise, it is an FN. On the basis that Tₘᵢₙ=10 s, detecting a seizure with duration equals to Tₘᵢₙ should be sufficient to conclude that a seizure is detected. The overlap duration can be computed as:

$${T}_{oνerlap} = \{ \begin{matrix}{d}_{end} - {d}_{start} & if {d}_{start} \geq {s}_{start} , {d}_{end} \leq {s}_{end} \\ {d}_{end} - {s}_{start} & if {d}_{start} < {s}_{start} , {d}_{end} \leq {s}_{end} \\ {s}_{end} - {d}_{start} & if {d}_{start} \geq {s}_{start} , {d}_{end} > {s}_{end} \\ {s}_{end} - {s}_{start} & otherwise .\end{matrix} \qquad ( 23 )$$

where Tₒᵥₑᵣₗₐₚ is the duration overlap between the detection window and the seizure window, dₛₜₐᵣₜ and d_{end} is the start and end time of the detected window, and sₛₜₐᵣₜ and s_{end} is the start and end time of the seizure window.

The TP and FN can be defined as:

$$TP = \{ \begin{matrix}1 & if {T}_{overlap} \geq {T}_{m i n} \\ 0 & otherwise .\end{matrix} \qquad ( 24 )$$

$$FN = \{ \begin{matrix}0 & if {T}_{overlap} \geq {T}_{m i n} \\ 1 & otherwise .\end{matrix} \qquad ( 25 )$$

Meanwhile, an FP_{missed} is recorded if the detection window failed to detect a seizure. Additionally, if a single detection window detected multiple seizure event within its window length, any interval between the seizures are treated as an FPₑᵥₑₙₜ. This is because it may not be sufficient to detect the seizures; detecting the correct number of seizures is also essential.

$${FP}_{missed} = \{ \begin{matrix}1 & if {T}_{overlap} \leq {T}_{m i n} \\ 0 & otherwise .\end{matrix} \qquad ( 26 )$$

$${FP}_{event} = \{ \begin{matrix}n - 1 & if {T}_{overlap} \geq {T}_{m i n} \\ 0 & otherwise .\end{matrix} \qquad ( 27 )$$

where n is the number of seizures detected by T_{detection}.

$${FP}_{total} = {FP}_{missed} + {FP}_{event} \qquad ( 28 )$$

Finally, when a TP occurs, the detection offset is the time difference between the start of the seizure event and the start of the detection event. The detection offset can be both positive (delay) and negative (early). If a detection overlaps with multiple seizure events, we only consider the detection offset first seizure. Likewise, if multiple detections overlap with a single seizure, we only consider the detection offset of the first detection. Only considering the first detection or seizure ensures that multiple offsets are not computed for the same detection or seizure. Hence, the detection offset can be defined as:

$${T}_{offset} = {d}_{start} - {s}_{start} + W + ⌊ \frac{{K}_{f}}{2} ⌋ ( W \times o ) , \qquad ( 29 )$$

where W are the duration of the window length, dₛₜₐᵣₜ is the time where the first seizure onset is detected, sₛₜₐᵣₜ is the starting time of the first seizure onset annotated by the expert, K_{f} is the filter length of the postprocessing module, and o is the overlap percentage.

We add the window length as the offset is constantly displaced by W, as the detector has to detect a minimum window length of W to determine if a seizure exists. Meanwhile, we include the second component to account for the nature of the seizure detector in this work. In the EEG-level postprocessing step, we applied a smoothen filter that utilized seizure information of several epochs ahead of time. The number of epochs required is equivalent to

$$⌊ \frac{{K}_{f}}{2} ⌋$$

epochs, where each successive epoch has a time difference of W×o seconds.

As a whole, the MOES is the middle point of the OVLP and TAES approaches. We set a fixed minimum duration for seizure detection, which is independent of the actual seizure duration. By doing so, we developed a metric that extracts the benefits of both OVLP and TAES, and combines them into a single metric.

### 1.2n EEG-Level Seizure Detection Performance Metrics

We measured the performance of the EEG-level seizure detection with the SEN, precision (PRE), false-positive per hour (FPR/h), and detection offset. The FPR/h is defined as:

$$FPR / h = \frac{FP}{Duration ( inhours )} , \qquad ( 30 )$$

where duration is in hours.

We note that the median FPR/h per file may be more appropriate as an EEG-level seizure detection metric, as it handles outliers much better than using mean. Similarly, we compute the median detection offset to account for the negative and positive offsets, as a mean may negate the negative and positive offsets, artificially boosting the results.

Finally, we noticed that most of the studies on EEG-level seizure detection do not report the PRE. However, PRE is a vital metric. For real-world real-time/offline seizure detection, PRE informs us of the fraction of correct alarms among all the alarms. The PRE information is critical in a clinical setting; if the PRE is poor, there are many false alarms among an actual seizure. In that case, most hospitals will be reluctant to deploy such systems, even if it has a high SEN, as the high FPs can lead to inefficiency in human resources and annoyance to the hospital staff and patients.

### 1.2o Training and Testing Cross-Validation

We trained the seizure detector on the TUH-SZ dataset and tested the trained model on the remaining five datasets for all EEG scales. For the TUH-SZ dataset, we performed 4-fold cross-validation (CV), which accounts for an equal split across different seizure types. For the remaining datasets, which are the testing datasets, we performed a leave-one-patient-out (LOPO) or a leave-one-file-out (LOFO) CV, whichever is applicable. The NVIDIA GeForce GTX1080 GPU machines and Keras 2.2.0 and TensorFlow 2.6.0 were deployed in this study.

### 1.2p Segment-Level Seizure Binary Classification: Machine Learning Model

Channel-level classification is performed on the channels in a multi-channel segment to obtain a list of probability outputs between [0,1]. With these probabilities, we extract features that applies for EEGs with any number of channels. With knowledge of the EEG channel location and their associated probabilities, we binned the channels into five scalp regions: frontal, central, occipital, parietal, and global (all the channels on the scalp). If the channel information is unavailable, all five scalp regions will be set as global to maintain the same number of regions for consistency during training and testing. Regional-based statistics features allow EEGs with any channels to be inputted, allowing flexibility of any type of EEGs to be classified. Finally, from each regions we extracted standard statistical features (mean, median, standard deviation, maximum, and minimum) and normalized histogram features (5 bins, range set at [0,1]), resulting ins in 5×10=50 features.

Prior to training of the machine learning classifiers, three data feature engineering processing steps are applied to the training data. First, any features with standard deviation of values ≤10⁻⁷ are rejected. Then, the features are normalized to a Normal Distribution N(0,1). Finally, Synthetic Minority Over-sampling Technique (SMOTE) (k=5) is applied to construct synthetic samples for balanced training⁶³. Finally, we employ different machine learning models to determine if the multi-channel segment contains seizure. We applied six learning classifiers in this study: Logistic Regression (LR)⁶⁴, Support Vector Machine (SVM)⁶⁵, Gradient Boosting (GB)⁶⁶, AdaBoost, (AB)⁶⁷, Random Forest (RF)⁶⁸, and XGBoost (XGB)⁶⁹. We determine the best model and hyperparameters with GridSearch Cross-Validation (CV).

### 1.2q Channel- and Segment-Level Seizure Classification Performance Metrics

The performance of the channel- and segment-level classification will be defined with the accuracy (ACC), balanced accuracy (BAC), sensitivity (SEN), specificity (SPE), and F1-score (F1). As the seizure and non-seizure classes are typically imbalanced, we evaluate the results mainly in BAC.

### 1.2r EEG-Level Seizure Detection Objective Metrics

To verify if the seizure detections are accurate, the detection must overlap with the actual annotated seizures. However, overlapping is simply the minimum criteria for a correct detection. Hence, the objective evaluation metrics must be adequately defined to compute the True Positive (TP), False Negative (FN), and False Positive (FP). Unfortunately, there is no standard objective evaluation metrics, as they are usually loosely defined. The common metrics deployed are the EBS, OVLP, and TAES [37]. Even so, these metrics contains imperfection and failed to reflect the correct requirement in clinical setting. Hence, in this work, we defined our objective evaluation metrics for EEG-level seizure detection performance.

### 1.3 Results

### 1.3a Channel- and Segment-Level Seizure Binary Seizure Classification Results

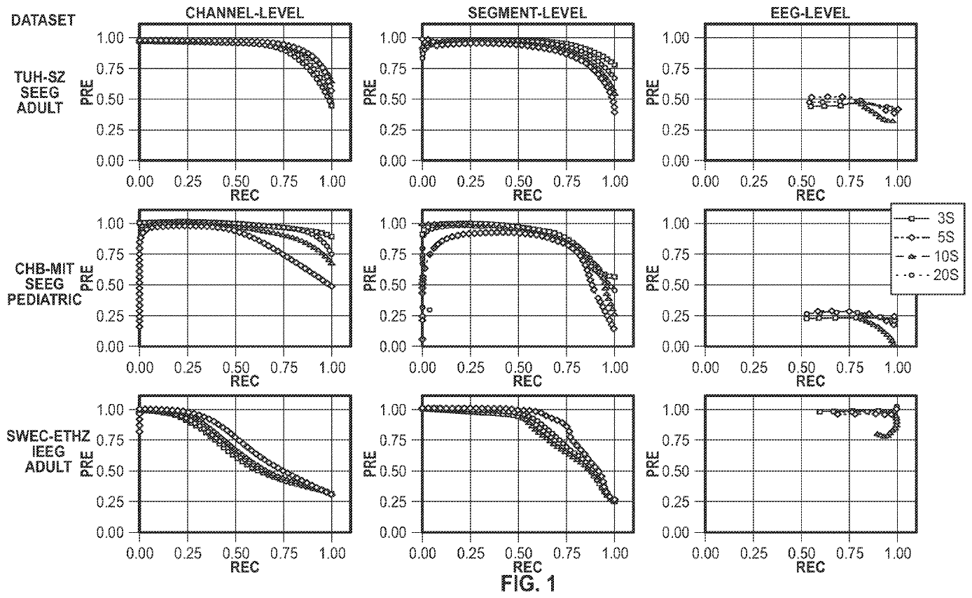

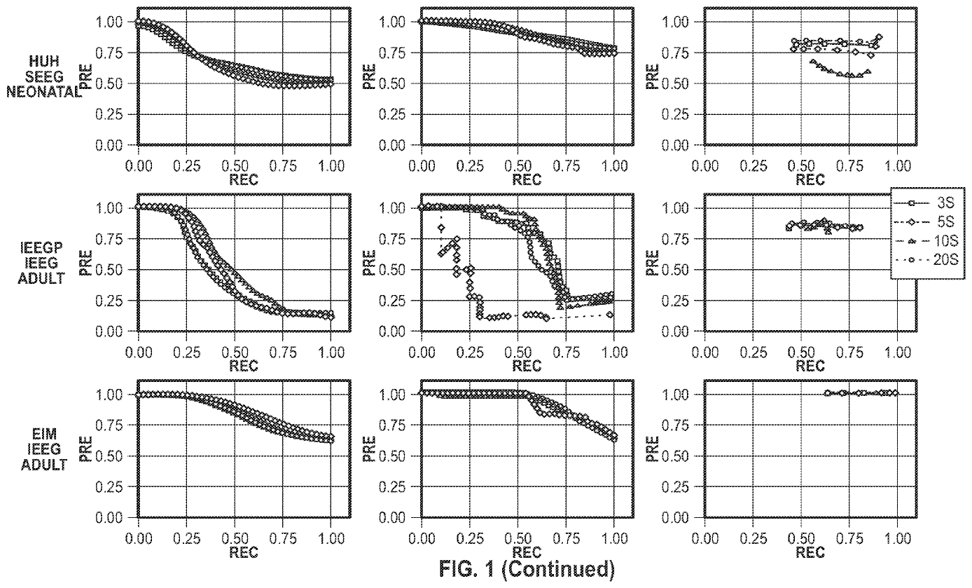

The channel- and segment-level seizure classification results for the six datasets are displayed in Table 1 and 2. We plot the precision-recall curve for the channel- and segment-level classification in FIG. 1. For both the channel- and segment-level seizure detection, the TUH-SZ dataset achieved high BAC, with excellent SEN and SPE across all window lengths. The results imply that the dataset is relatively clean of artifacts and rather suitable for training the channel- and segment-level seizure detector. As the seizure detector begins with channel-level detection, the channel-level detection must be accurate, or the error may be amplified and passed to the posterior modules. The CNN+Transformer+BM model obtained the best results, followed by the CNN+BM model and the CNN model. The results are expected as the transformers and BM can help better generalize classification for different patients.

Across the other five datasets, the CHB-MIT, SWEC-ETHZ, and EIM datasets achieve decent performance for channel-level classification, while the HUH and IEEGP datasets achieve poor results. The poor results is due to the lack of channel-level seizure annotation for these datasets, resulting in many non-seizure channels being treated as seizure channels despite not containing any. Without filtering the non-seizure affected channels during testing, poor BAC scores are expected. However, when looking at the segment-level results, the CHB-MIT, SWEC-ETHZ, HUH (for short window length), IEEGP (for short window length), and EIM datasets all achieved excellent results for different window length and model combinations. Overall, the segment-level detector appeared to perform decently, even if the results from the channel-level detector may be poor. Hence, it is demonstrated that the segment-level detector helps to reduce error induced from the channel-level output, leading to good BAC.

Overall, the channel- and segment-level results demonstrated the usefulness of a cascading approach; the performance improved across the successive module. Despite differences across EEGs from different datasets, not limited to signal gain, seizure type, and EEG recording system, the multi-cascade system can generalize the system better than traditional approaches. Generally, the model with transformer and BM yielded the best overall results across the five datasets, followed by the CNN+BM model and the CNN model.

Finally, evaluating the systems based on different window lengths, the TUH-SZ, CHB-MIT, SWEC-ETHZ, and EIM datasets have higher SEN for a longer window length (10, 20 s). Meanwhile, the HUH and IEEGP dataset achieved better SEN for a shorter window length (3, 5 s). The differences can be attributed to each dataset containing different seizure types and duration. For instance, the results may be skewed towards more prolonged seizures, where more segments are extracted during training and testing, placing greater weights in detecting those segments. Hence, this does not reflect the final output of the seizure detector. Ultimately, comparing the performance using EEG-level seizure detection results may be more appropriate.

### EEG-Level Seizure Detection Results

We computed the EEG-level seizure detection results with the MOES metric (see Table 4) and displayed the overall SEN, PRE, median FPR/h, and the median detection offset. Also, we plot the PR-curve in FIG. 1. As the EEG-level seizure detection is not a binary classification, the PR-curve will be discontinuous at the extreme points. Hence, to plot the PR-curve, we varied the segment-level threshold, with other parameters maintained constant. Finally, to compare our results with different works that deployed different seizure evaluation metrics, we also display the results for the transformer with BM loss, evaluated with MOES, OVLP, and TAES in Table 3.

The TUH-SZ dataset achieved high SEN with decent PRE while maintaining a median FPR/h of 0 for the EEG-level seizure detection. A median FPR/h of 0 implies that more than 50% of the EEGs do not contain any FPs at all. Generally, the transformer with BM loss produced the best results, followed by the CNN with BM loss, and lastly, the CNN. While the SEN is similar across the three models, the transformer with BM loss has a much better PRE, necessary for real-life deployment.

Next, we evaluated the five other datasets with the seizure detector via transfer learning. The CHB-MIT, SWEC-ETHZ, and EIM datasets achieved excellent results with high SEN and modest PRE while maintaining a low FPR/h. Meanwhile, the HUH and IEEGP datasets achieved modest SEN but with high PRE and low FPR/h. Focusing mainly on the PRE, the SWEC-ETHZ, HUH, IEEGP, and EIM datasets achieved high PRE.

Next, we focused on the median offset results. The majority of the offsets are negative, which at first glance implies possible seizure forecasting. However, this may be far from the truth. The negative offsets are due to several factors, mainly due to offline analysis and not seizure forecasting.
[topsep=0pt,itemsep=0ex,partopsep=1ex,parsep=1ex]
   1. Firstly, the seizure detection is performed offline. In other words, the entire EEG was input into the detector to perform seizure detection. As a result, we have access to the posterior information of any potential seizure onset. By deploying posterior information (via median filter and seizure clusters) of any seizure onset to perform seizure detection, we effectively did not detect seizure at the registered onset time but only backtracked after several additional epochs of that seizure onset. In other words, we should include the time window of those additional epochs to the offset computed here if one were to deploy the current seizure detector in a real-time system.
   2. Secondly, with a median filter, the high seizure probabilities clusters can be propagated and elongated on both ends for any seizure event, forwarding the start time and delaying the end time of the seizure event. Hence, the detection onset is pushed forward due to the postprocessing module, resulting in a negative value that does not imply forecasting.
   3. Thirdly, the seizure annotation may not be perfectly aligned with the actual seizure event. For example, the annotator might annotate the seizure in the later stages, where the morphology of the seizure is more mature and well developed. On the other hand, the annotator might annotate the seizure much earlier, where the seizure morphology just started to develop. In both cases, the annotation may unknowingly generate a negative or positive offset. It is to note, however, that this factor should not impact seizure detection significantly.
   4. Lastly, the seizure detector performs seizure detection in a discrete manner. Ideally, the overlap percentage of the sliding window should be as small as possible, corresponding to 1 sample of the signal. However, this is too computationally intensive. Hence, we set the slider to a fixed duration of is. As a result, some offset differences will occur if the starting seizure annotation is not an integer value. Nonetheless, this issue may result in both negative and positive offset duration.

Next, as the transformer yields the best results, we compare the results evaluated with MOES, OVLP, and TAES by the transformer in Table 3, to determine the performance difference with different evaluation metrics. The OVLP approach yields the best results, while the TAES approach yields the worst results. The results achieved by MOES lie in between OVLP and TAES. On average, the results recorded via MOES is much closer to those using OVLP. On the other hand, the results by TAES achieved the poorest results by far.

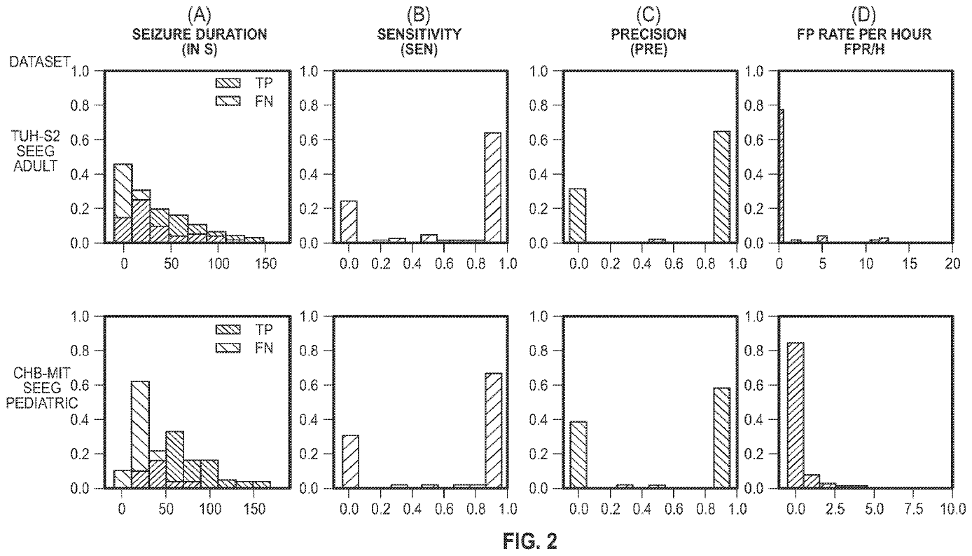

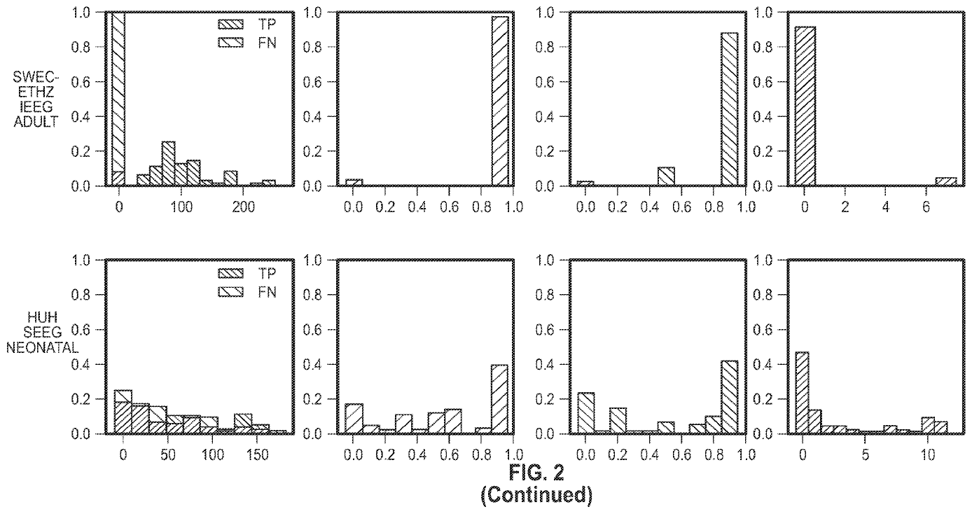

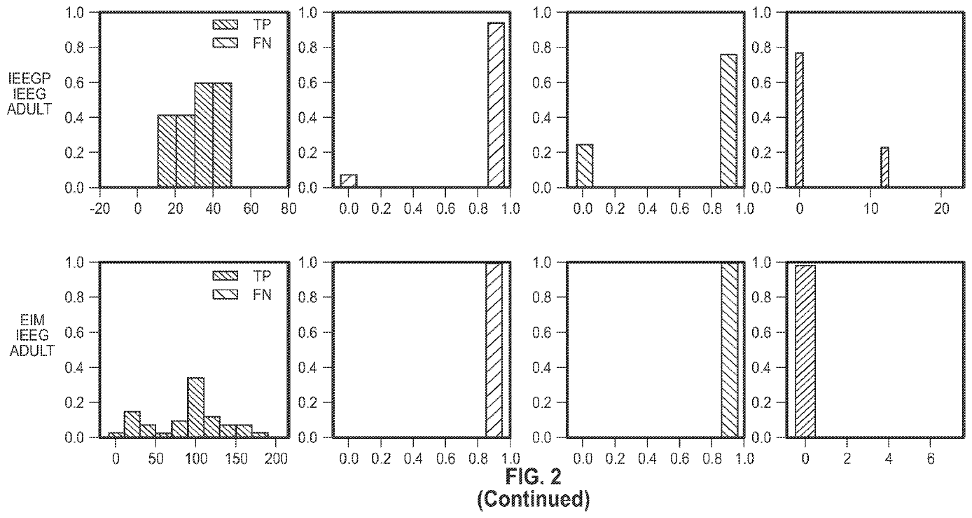

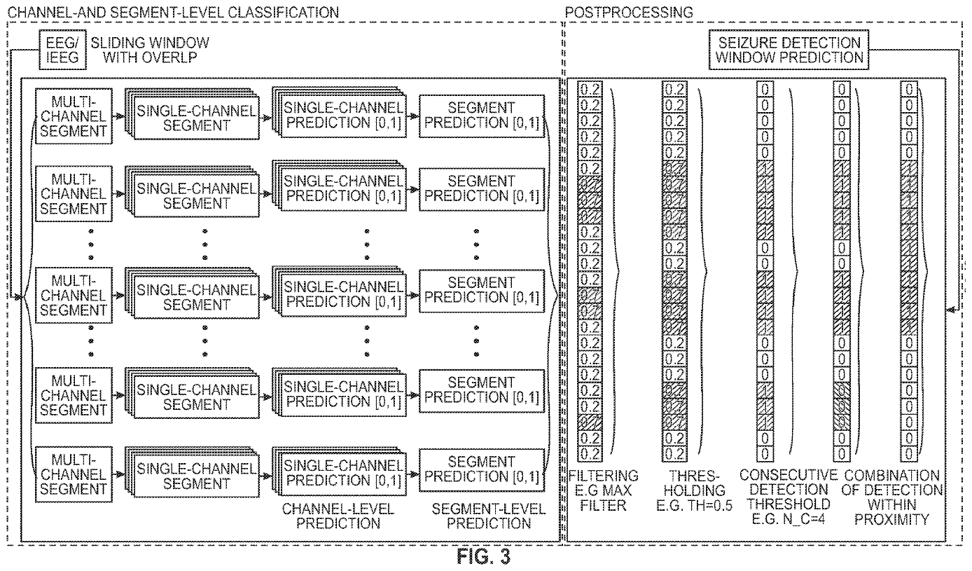

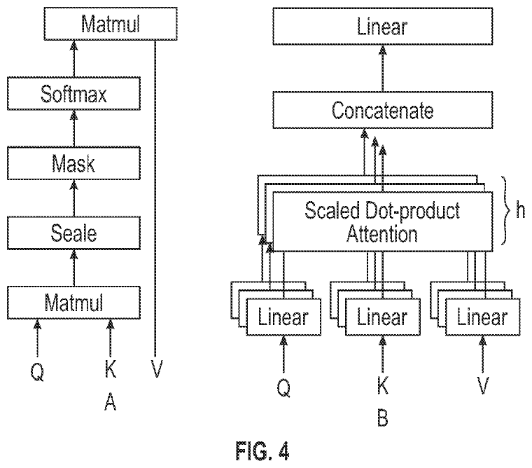

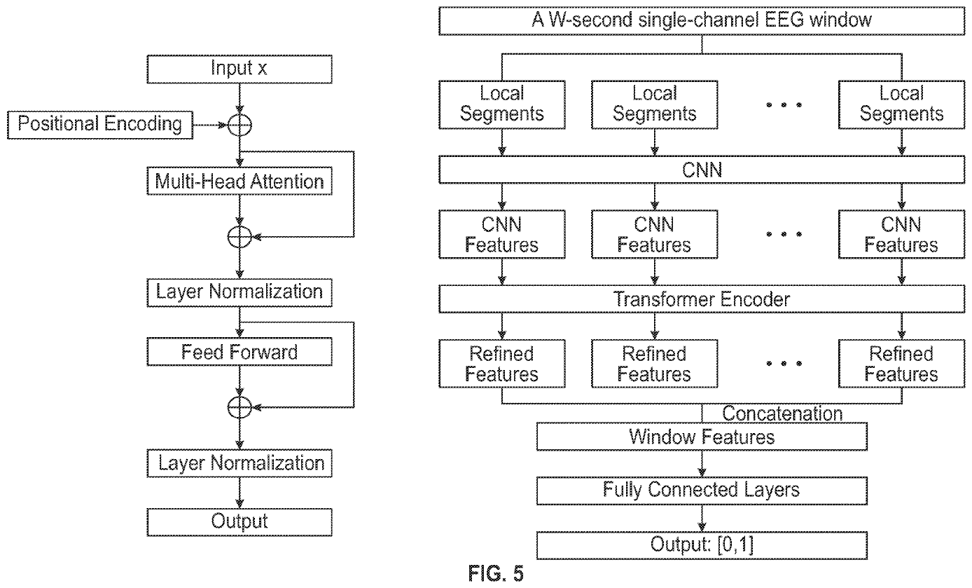

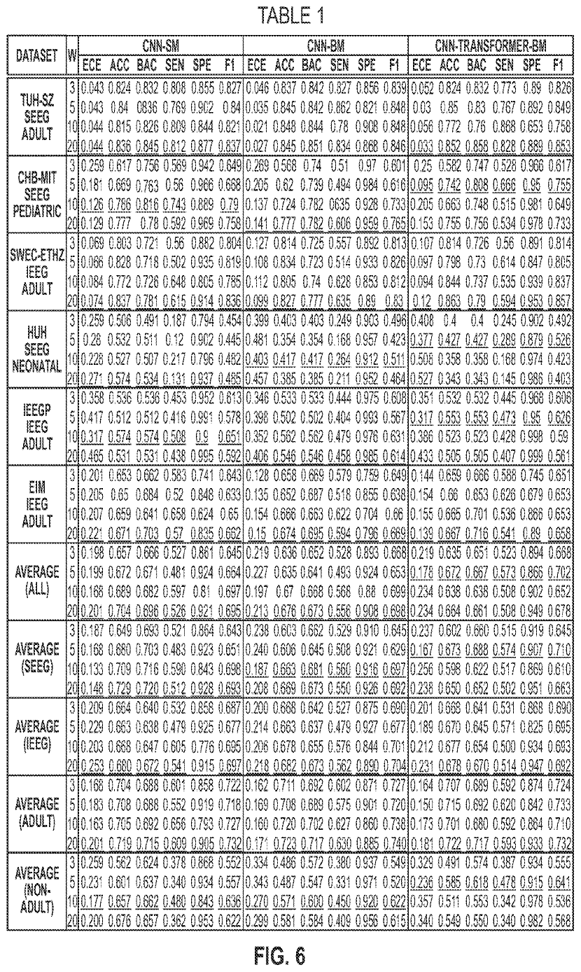

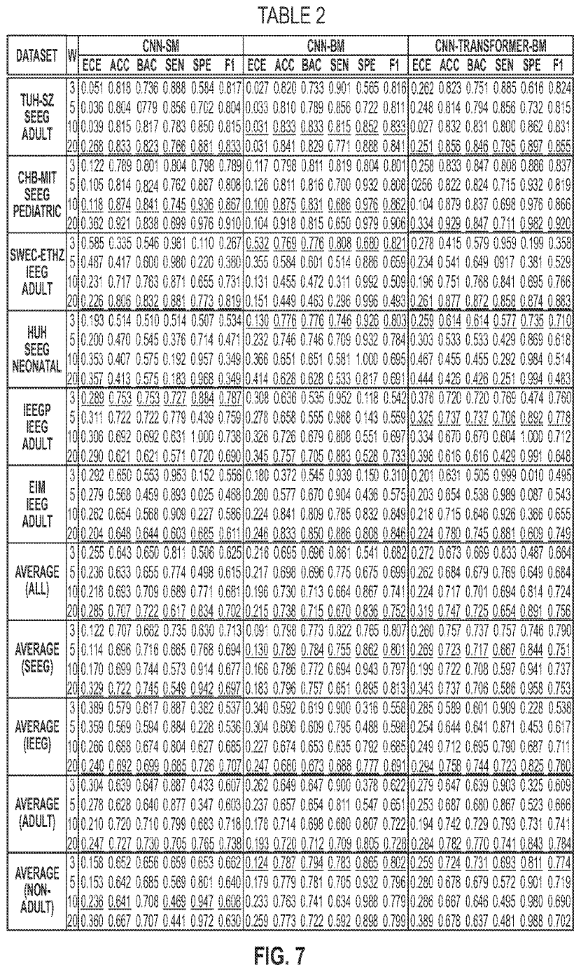

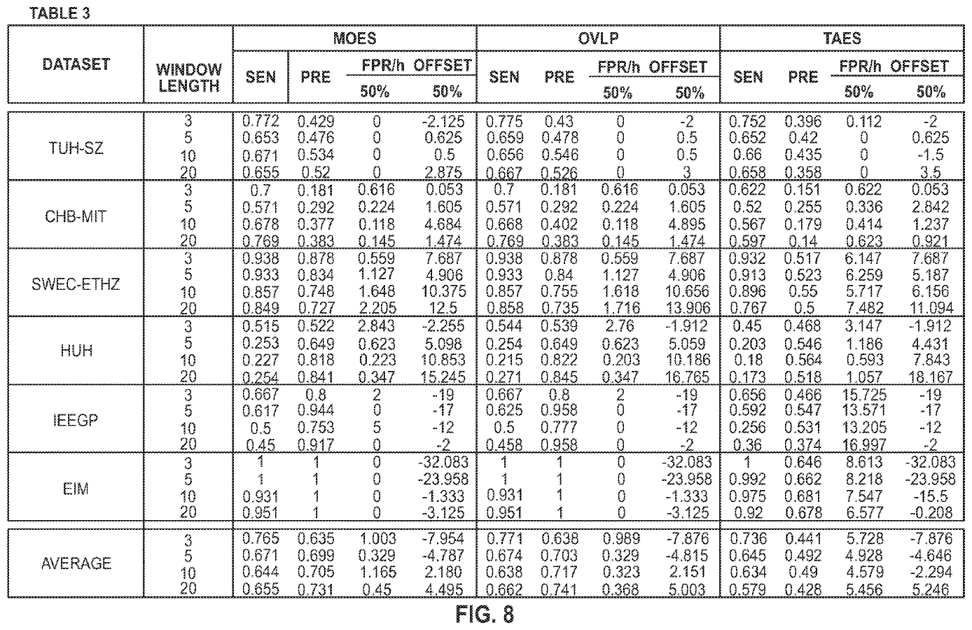

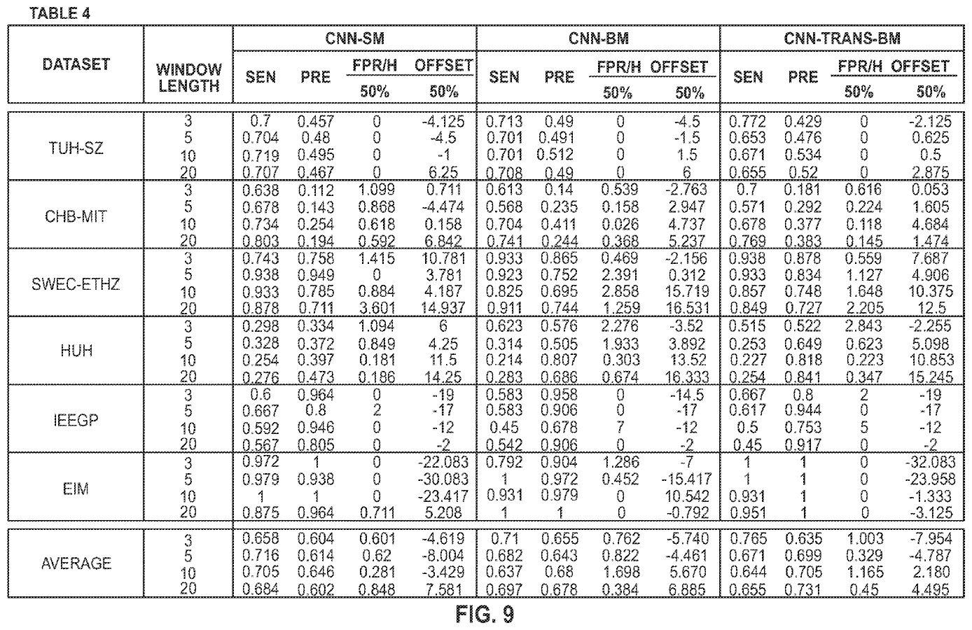

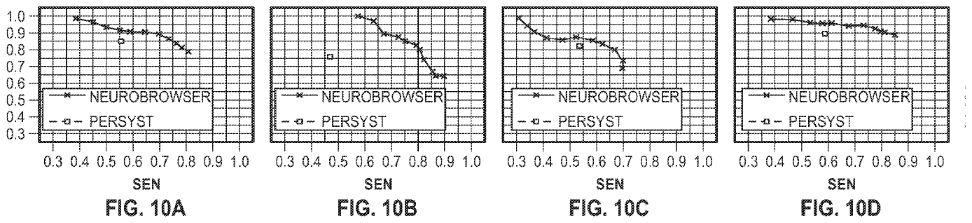

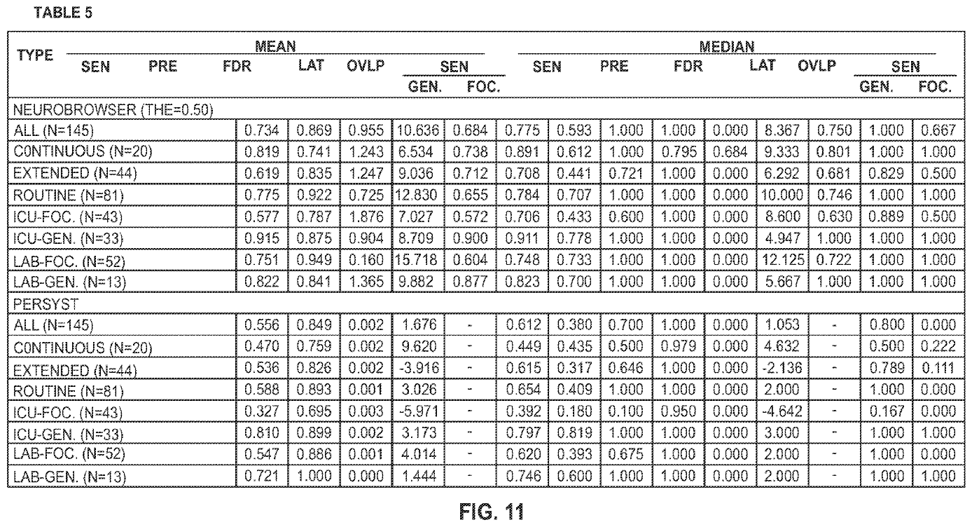

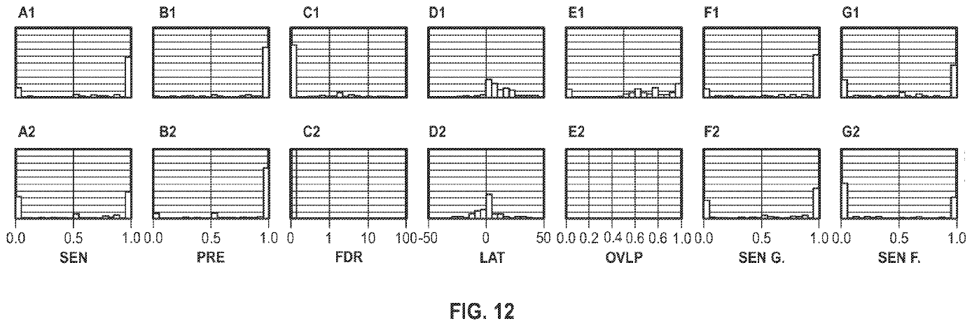

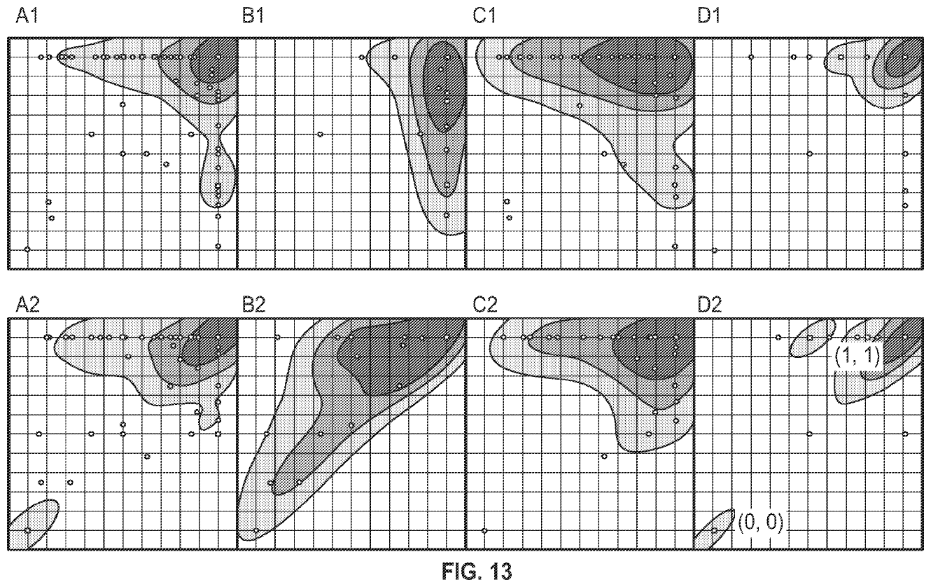

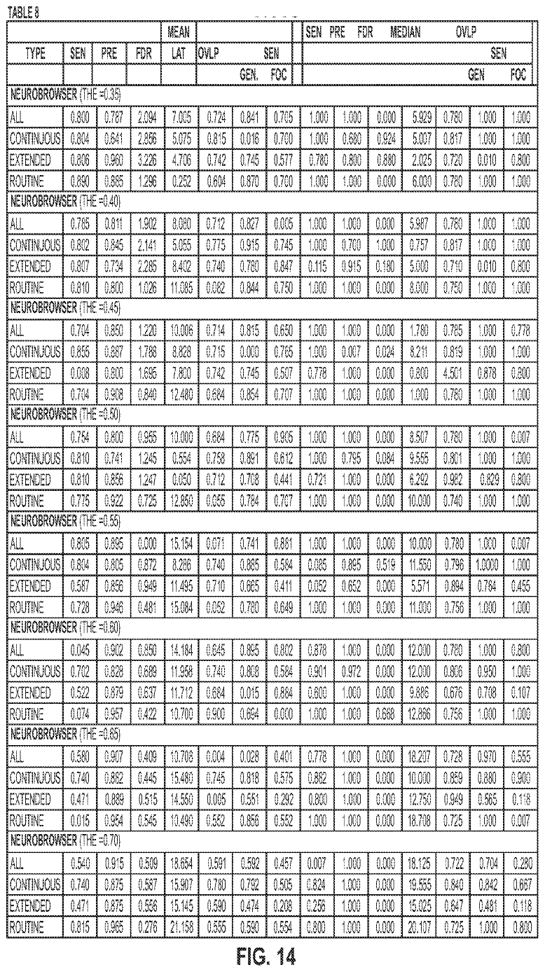

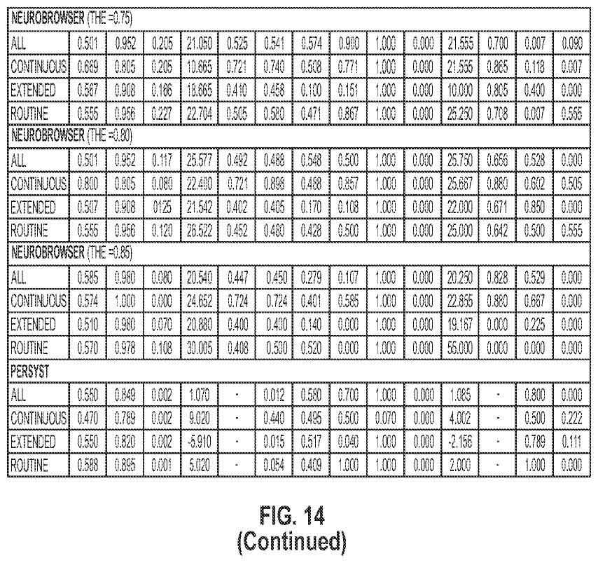

Finally, we visualized the TP and FN of seizures with various duration, the SEN, PRE, and FPR/h histogram distribution of all EEG files combined across the datasets in FIG. 2. The plot of TP and FN of seizures with various duration revealed that a more prolonged seizure is easier to detect, while a shorter seizure is more challenging to identify. Meanwhile, the SEN and PRE for most files are high, indicating that the detector can detect seizures in most files with high PRE and low false detection. Lastly, most files have 0 FPR/h, with only a small percentage of files with FPR/h greater than 0. Hence, the detector did not make any mistakes in most files, leading to a median FPR/h of 0.

As such, we showed that the proposed seizure detector model could be applied across multiple datasets without retraining. Hence, the seizure detector illustrated in this work can perform non-patient-specific seizure detection across different EEGs/iEEGs that contain different patient-specific decently via transfer learning.

### Example 2

### 2.1 NeuroBrowser Seizure Detector and the Test Dataset

In this example, benchmark the performance of the NeuroBrowser (NB) automated seizure detector against a commercial seizure detection software—Persyst 14, on 145 adult EEGs was benchmarked. In this benchmark, the vanilla version of the XGBoost-based segment-level classifier with regional features in the NeuroBrowser seizure detector was used. The dataset used is a subset of the full NNI dataset which comprises 145 EEGs in total, of which 20 are continuous EEGs, 44 are extended EEGs and 81 routine EEGs.

To benchmark the performance of NeuroBrowser, both seizure detectors were run on the 145 test EEGs and calculate the statistics of the detected seizures. The NeuroBrowser seizure detector gives as output the start and end times of the detected seizures, as well as the EEG channels that are affected in each seizure. The output from Persyst is in the form of CSV files, which indicates whether or not a seizure exists at each timestamp. In contrast to NeuroBrowser however, Persyst does not provide information as to the channels in which a seizure is detected. The outputs are converted to a common format for benchmarking purposes. We compare the performance of the two seizure detectors using metrics including the sensitivity, precision, false detection rate and latency.

### 2.2 Comparison of Detection Performance Between NeuroBrowser and Persyst

FIG. 10 shows the precision-recall (PR) curve for the NeuroBrowser seizure detector with the performance of Persyst as benchmark. It can be observed from the figures that with an appropriate postprocessing threshold, the performance of NeuroBrowser significantly outperforms that of Persyst. In Table 5 the detection metrics of NeuroBrowser at postprocessing threshold ThE=0.5 and Persyst (see Table 8 for the detection metrics of NeuroBrowser at different postprocessing thresholds) are shown. From the table, one can observe that NeuroBrowser is superior to Persyst in every metric except latency and false detection rate (FDR). The channel overlap metric is not available for Persyst as the channel-level predictions are missing from its output CSV files.

Overall, NeuroBrowser achieves a significantly higher mean overall sensitivity and precision of 0.734 and 0.869 respectively. Although the false detection rate of NeuroBrowser is higher than that of Persyst, NeuroBrowser is detecting many more seizures correctly as evident in the high sensitivity and precision.

The NeuroBrowser seizure detector is also much more sensitive to focal seizures than Persyst, with NeuroBrowser detecting 59.3% of all focal seizures within each EEG on average compared to 38.0% for Persyst. We observe a marked difference when we compare the median sensitivity towards focal seizures between the two detectors, which stands at 66.7 percentage points. NeuroBrowser is also more sensitive, although less so compared to focal seizures, towards generalised seizures, with a sensitivity of 0.775 compared to 0.612 for Persyst.

One metric in which Persyst performs better on average is latency. The average latency of the detections is 10.6 seconds for NeuroBrowser and 1.7 seconds for Persyst. However, if we compare the distributions of the average latencies across EEGs with both NeuroBrowser and Persyst (see FIG. 12), we observe that the the lower average latency for Persyst is partly due to the symmetric distribution about the zero-point. These statistics may well also be incomparable as NeuroBrowser is detecting 0.32 times more seizures than Persyst.

From the distributions of the metrics in FIG. 12, it was observed that the performance of Persyst varies significantly across EEGs as seen from bimodal distributions of the detection metrics. In particular, Persyst failed to detect any seizures in 31% of the 145 test EEGs. It also failed to detect any generalised and focal seizures in 21% and 22% of the test EEGs respectively. NeuroBrowser on the other hand displays more predictable behaviour and detects seizures with high sensitivity and precision most of the time with few outliers as can be observed from FIG. 13.

In conclusion, from a practical standpoint, NeuroBrowser outperforms Persyst significantly as it is able to detect more seizures and does so more accurately with a small tradeoff in false detection rate.

### EXEMPLARY EMBODIMENTS

The following embodiments are set forth as illustrative examples of aspects and objects of the invention. Each of these embodiments incorporates any element or multiple elements of the Examples set forth herein in any permutation or order.

In various embodiments, there is provided an automated method of seizure detection using a method selected from scalp electroencephalogram (EEG), intracranial EEG (iEEG) and a combination thereof, the method comprising:
(a) applying a transformer with a convolutional neural network (CNN) and a belief matching (BM) loss to acquire subject data from at least one single-channel segment (channel-level), acquiring channel-level data;
(b) extracting from the channel-level data at least two regional features, thereby acquiring data from multi-channel segments (segment level);
(c) from the multi-channel segment data, predicting seizure probability in the multi-channel segments;
(d) repeating (c) at least once, acquiring successive segment-level outputs; and
(e) applying at least one convolutional-based postprocessing module on the successive segment-level outputs, thereby detecting the seizure.

In various embodiments, there is provided a method, wherein the transformer is a deep learning model based on attention mechanism.

In various embodiments, there is provided a method, wherein the attention mechanism is self-attention.

In various embodiments, there is provided a method, wherein loss of order information at the channel-level is mitigated by applying positional encoding to channel-level input data.

In various embodiments, there is provided a method, wherein subject data are acquired from at least 10 channel segments, at least 15 channel segments, or at least 20 channel segments.

In various embodiments, there is provided a method, wherein subject data are acquired from at least 21 channel segments.

In various embodiments, there is provided a method, wherein the channel-level data are data from an electroencephalogram.

In various embodiments, there is provided a method, wherein the channel-level data yields seizure probabilities for each channel.

In various embodiments, there is provided a method, wherein the seizure probabilities are arranged into regions according to the scalp topology: frontal, central, occipital, and parietal. Besides those four local regions, we also define a “global” region containing.

In various embodiments, there is provided a method, wherein the seizure probabilities are further arranged into a “global” region containing all channels.

In various embodiments, there is provided a method, further comprising extracting from each region at least one of seven statistical features: mean, median, standard deviation, maximum value, minimum value, and value at 25% and 75% percentile, thereby forming a feature set.

In various embodiments, there is provided a method, wherein there are five regions and 5×7=35 features are extracted.

In various embodiments, there is provided a method, further comprising computing from all channel-level outputs normalized histogram features (5 bins, range [0,1]) and including them into the feature set, bringing the total features in the feature set to 40.

In various embodiments, there is provided a method, wherein the repeating in (d) is performed for all EEG segments.

In various embodiments, there is provided a method, wherein (e) comprises: applying at least one 1D smoothing filter, thereby removing from the data isolated seizure detections (e.g., false positives), and smoothing regions with significant confidence variations, and stabilizing the detections.

In various embodiments, there is provided a method, wherein (e) further comprises: following smoothing, thresholding to the seizure probabilities to round them to zeros (seizure-free) or ones (seizure).

In various embodiments, there is provided a method, wherein a threshold value θ∈{0.1, 0.2, 0.3, 0.4. 0.5, 0.6. 07, 0.8, or 0.9} is utilized.

In various embodiments, there is provided a method, further comprising: following thresholding, identifying consecutive is of length smaller than Nc, and replacing the identified is with 0 s, thereby removing short detections, leading to fewer false positives and more false negatives, as the system may miss short seizures.

In various embodiments, there is provided a method, wherein Nc∈{1, 2, 3, 4, 5, 6, 7, 8. 9. 20, 11, 12, 13, 14, 15, 16, 17, 18, 19, or 20}.

In various embodiments, there is provided a method, wherein following the replacing remaining sequences of consecutive 1 s are identified, and the start and end time of the consecutive 1 s is identified, thereby resulting in a final output of the EEG-level seizure detector being the start and end times of the detected seizures.

The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

In various embodiments, there is provided a method, wherein Nc∈{1, 2, 3, 4, 5, 6, 7, 8. 9. 20, 11, 12, 13, 14, 15, 16, 17, 18, 19, or 20}.

In various embodiments, there is provided a method, wherein following the replacing remaining sequences of consecutive is are identified, and the start and end time of the consecutive 1 s is identified, thereby resulting in a final output of the EEG-level seizure detector being the start and end times of the detected seizures.

The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

### REFERENCES

[1] Ali Kareem Abbas, Ghasem Azemi, Samin Ravanshadi, and Amir Omidvarnia. An eeg-based methodology for the estimation of functional brain connectivity networks: Application to the analysis of newborn eeg seizure. Biomedical Signal Processing and Control, 63:102229, 2021.
[2] David Ahmedt-Aristizabal, Tharindu Fernando, Simon Denman, Lars Petersson, Matthew J Aburn, and Clinton Fookes. Neural memory networks for seizure type classification. In 2020 42nd Annual International Conference of the IEEE Engineering in Medicine & Biology Society (EMBC), pages 569-575. IEEE, 2020.
[3] Amir H Ansari, Perumpillichira J Cherian, Alexander Caicedo, Gunnar Naulaers, Maarten De Vos, and Sabine Van Huffel. Neonatal seizure detection using deep convolutional neural networks. International journal of neuralsystems, 29(04):1850011, 2019.
[4] Umar Asif, Subhrajit Roy, Jianbin Tang, and Stefan Harrer. Seizurenet: a deep convolutional neural network for accurate seizure type classification and seizure detection. arXiv preprint arXiv:1903.03232, 2019.
[5] K P Ayodele, W O Ikezogwo, M A Komolafe, and P Ogunbona. Supervised domain generalization for integration of disparate scalp eeg datasets for automatic epileptic seizure detection. Computers in Biology and Medicine, 120:103757, 2020.
[6] Steven Baldassano, Drausin Wulsin, Hoameng Ung, Tyler Blevins, Mesha-Gay Brown, Emily Fox, and Brian Litt. A novel seizure detection algorithm informed by hidden markov model event states. Journal of neural engineering, 13(3):036011, 2016.
[7] Steven Baldassano, Xuelong Zhao, Benjamin Brinkmann, Vaclav Kremen, John Bernabei, Mark Cook, Timothy Denison, Gregory Worrell, and Brian Litt. Cloud computing for seizure detection in implanted neural devices. Journal of neural engineering, 16(2):026016, 2019.
[8] Jimmy Lei Ba, Jamie Ryan Kiros, and Geoffrey E Hinton. Layer normalization. arXiv preprint arXiv: 1607.06450, 2016.
[9] Anne T Berg. Risk of recurrence after a first unprovoked seizure. Epilepsia, 49:13-18, 2008.
[10] Abhijeet Bhattacharya, Tanmay Baweja, and SPK Karri. Epileptic seizure prediction using deep transformer model. International Journal of Neural Systems, page 2150058, 2021.
[11] Abhijit Bhattacharyya and Ram Bilas Pachori. A multivariate approach for patient-specific eeg seizure detection using empirical wavelet transform. IEEE Transactions on Biomedical Engineering, 64(9):2003-2015, 2017.
[12] David M Blei, Alp Kucukelbir, and Jon D McAuliffe. Variational inference: A review for statisticians. Journal of the American statistical Association, 112(518):859-877, 2017.
[13] Charles Blundell, Julien Cornebise, Koray Kavukcuoglu, and Daan Wierstra. Weight uncertainty in neural network. In International Conference on Machine Learning, pages 1613-1622. PMLR, 2015.
[14] Benjamin H Brinkmann, Joost Wagenaar, Drew Abbot, Phillip Adkins, Simone C Bosshard, Min Chen, Quang M Tieng, Jialune He, F J Muñoz-Almaraz, Paloma Botella-Rocamora, et al. Crowdsourcing reproducible seizure forecasting in human and canine epilepsy. Brain, 139(6):1713-1722, 2016.
[15] Jeffrey W Britton, Lauren C Frey, J L Hopp, P Korb, M Z Koubeissi, W E Lievens, E M Pestana-Knight, and E K Louis St. Electroencephalography (EFC): An introductory text and alias of normal and abnormal findings in adults, children, and infants. American Epilepsy Society, Chicago, 2016.
[16] Alessio Burrello, Kaspar Schindler, Luca Benini, and Abbas Rahimi. Hyperdimensional computing with local binary patterns: one-shot learning of seizure onset and identification of ictogenic brain regions using short-time ieeg recordings. IEEE Transactions on Biomedical Engineering, 67(2):601-613, 2019.
[17] Christos Chatzichristos, J Dan, A Mundanad Narayanan, Nick Seeuws, K Vandecasteele, M De Vos, A Bertrand, and S Van Huffel. Epileptic seizure detection in eeg via fusion of multi-view attention-gated u-net deep neural networks. In Proceedings of the IEEE Signal Processing in Medicine and Biology Symposium (SPMB), page 7, 2020.
[18] Nitesh V Chawla, Kevin W Bowyer, Lawrence O Hall, and W Philip Kegelmeyer. Smote: synthetic minority over-sampling technique. Journal of artificial intelligence research, 16:321-357, 2002.
[19] Ian C Covert, Balu Krishnan, Imad Najm, Jiening Zhan, Matthew Shore, John Hixson, and Ming Jack Po. Temporal graph convolutional networks for automatic seizure detection. In Machine Learning for Healthcare Conference, pages 160-180. PMLR, 2019.
[20] A Einizade, M Mozafari, S Hajipour Sardouie, S Nasiri, and G Clifford. A deep learning-based method for automatic detection of epileptic seizure in a dataset with both generalized and focal seizure types. In 2020 IEEE Signal Processing in Medicine and Biology Symposium (SPUB), pages 1-6. IEEE, 2020.
[21] Themis P Exarchos, Alexandros T Tzallas, Dimitrios I Fotiadis, Spiros Konitsiotis, and Sotirios Giannopoulos. Eeg transient event detection and classification using association rules. IEEE Transactions on Information Technology in Biomedicine, 10(3):451-457, 2006.
[22] Fred F Ferri. Ferri's Clinical Advisor 2020 E-Book: 5 Books in 1. Elsevier Health Sciences, 2019.
[23] F Firbass, P Ossenblok, M Hartmann, H Perko, A M Skupch, G Lindinger, L Elezi, E Pataraia, A J Colon, C Baumgartner, et al. Prospective multi-center study of an automatic online seizure detection system for epilepsy monitoring units. Clinical Neurophysiology, 126(6):1124-1131, 2015.
[24] Jonas Gehring, Michael Auli, David Grangier, Denis Yarats, and Yann N Dauphin. Convolutional sequence to sequence learning. In International Conference on Machine Learning, pages 1243-1252. PMLR, 2017.
[25] I Geut, S Weenink, I L H Knottnerus, and Michel J A M van Putten. Detecting interictal discharges in first seizure patients: ambulatory eeg or eeg after sleep deprivation?Seizure, 51:52-54, 2017.
[26] Meysam Golmohammadi, Amir Hossein Harati Nejad Torbati, Silvia Lopez de Diego, Tyad Obeid, and Joseph Picone. Automatic analysis of eegs using big data and hybrid deep learning architectures. Frontiers in human neuroscience, 13:76, 2019.
[27] Meysam Golmohammadi, Saeedeh Ziyabari, Vinit Shah, Silvia Lopez de Diego, Iyad Obeid, and Joseph Picone. Deep architectures for automated seizure detection in scalp eegs. arXiv preprint arXiv: 1712.09776, 2017.
[28] Catalina Gómez, Pablo Arbeláez, Miguel Navarrete, Catalina Alvarado-Rojas, Michel Le Van Quyen, and Mario Valderrama. Automatic seizure detection based on imaged-eeg signals through fully convolutional networks. Scientific reports, 10(1):1-13, 2020.
[29] Chuan Guo, Geoff Pleiss, Yu Sun, and Kilian Q Weinberger. On calibration of modern neural networks. In International Conference on Machine Learning, pages 1321-1330. PMLR, 2017.
[30] W Allen Hauser. Seizure disorders: the changes with age. Epilepsia, 33:6-14, 1992.
[31] Tomas Iešmantas and Robertas Alzbutas. Convolutional neural network for detection and classification of seizures in clinical data. Medical & Biological Engineering & Computing, 58(9):1919-1932, 2020.
[32] Viktor K Jirsa, William C Stacey, Pascale P Quilichini, Anton I Ivanov, and Christophe Bernard. On the nature of seizure dynamics. Brain, 137(8):2210-2230, 2014.
[33] Taejong Joo, Uijung Chung, and Min-Gwan Seo. Being bayesian about categorical probability. In International Conference on Machine Learning, pages 4950-4961. PMLR, 2020.
[34] Muhammad Kaleem, Dharmendra Gurve, Aziz Guergachi, and Sridhar Krishnan. Patient-specific seizure detection in long-term eeg using signal-derived empirical mode decomposition (emd)-based dictionary approach. Journal of neural engineering, 15(5):056004, 2018.
[35] Thomas N Kipf and Max Welling. Semi-supervised classification with graph convolutional networks. arXiv preprint arXiv:1609.02907, 2016.
[36] Demetres Kostas, Stephane Aroca-Ouellette, and Frank Rudzicz. Bendr: using transformers and a contrastive self-supervised learning task to learn from massive amounts of eeg data. arXiv preprint arXiv:2101.12037, 2021.
[37] Qi Lian, Yu Qi, Gang Pan, and Yueming Wang. Learning graph in graph convolutional neural networks for robust seizure prediction. Journal of neural engineering, 17(3):035004, 2020.
[38] Zhouhan Lin, Minwei Feng, Cicero Nogueira dos Santos, Mo Yu, Bing Xiang, Bowen Zhou, and Yoshua Bengio. A structured self-attentive sentence embedding. arXiv preprint arXiv:1703.03130, 2017.
[39] Tennison Liu, Nhan Duy Truong, Armin Nikpour, Luping Zhou, and Omid Kavehei. Epileptic seizure classification with symmetric and hybrid bilinear models. IEEE journal of biomedical and health informatics, 24(10):2844-2851, 2020.
[40] Adam Li, Chester Huynh, Zachary Fitzgerald, Iahn Cajigas, Damian Brusko, Angel Claudio, Jonathan Jagid, Andres Kanner, Jennifer Hopp, Stephanie Chen, et al. Neural fragility as an eeg marker of the seizure onset zone. bioRxiv, page 862797, 2021.
[41] Amirsalar Mansouri, Sanjay P Singh, and Khalid Sayood. Online eeg seizure detection and localization. Algorithms, 12(9):176, 2019.
[42] Karl E Misulis and E Lee Murray. Essentials of Hospital Neurology. Oxford University Press, 2017.
[43] Florian Mormann, Ralph G Andrzejak, Christian E Elger, and Klaus Lehnertz. Seizure prediction: the long and winding road. Brain, 130(2):314-333, 2007.
[44] Petr Nejedly, Vaclav Kremen, Vladimir Sladky, Mona Nasseri, Hari Guragain, Petr Klimes, Jan Cimbalnik, Yogatheesan Varatharajah, Benjamin H Brinkmann, and Gregory A Worrell. Deep-learning for seizure forecasting in canines with epilepsy. Journal of neural engineering, 16(3):036031, 2019.
[45] Alison Shea, Gordon Lightbody, Geraldine Boylan, and Andriy Temko. Neonatal seizure detection from raw multi-channel eeg using a fully convolutional architecture. Neural Networks, 123:12-25, 2020.
[46] Maryam Odabaee, Walter J Freeman, Paul B Colditz, Ceon Ramon, and Sampsa Vanhatalo. Spatial patterning of the neonatal eeg suggests a need for a high number of electrodes. Neuroimage, 68:229-235, 2013.
[47] Jihun Oh, Kyunghyun Cho, and Joan Bruna. Advancing graphsage with a data-driven node sampling. arXiv preprint arXiv:1904.12935, 2019.
[48] World Health Organization, Global Campaign against Epilepsy, Programme for Neurological Diseases, Neuroscience (World Health Organization), International Bureau for Epilepsy, World Health Organization. Department of Mental Health, Substance Abuse, International Bureau of Epilepsy, and International League against Epilepsy. Atlas: epilepsy care in the world. World Health Organization, 2005.
[49] Tim Pearce, Alexandra Brintrup, and Jun Zhu. Understanding softmax confidence and uncertainty. arXiv preprint arXiv:2106.04972, 2021.
[50] J Pedoeem, S Abittan, G Bar Yosef, and S Keene. Tabs: Transformer based seizure detection. In 2020 IEEE Signal Processing in Medicine and Biology Symposium (SPMB), pages 1-6. IEEE, 2020.
[51] Wei Yan Peh, John Thomas, Elham Bagheri, Rima Chaudhari, Sagar Karia, Rahul Rathakrishnan, Vinay Saini, Nilesh Shah, Rohit Srivastava, Yee-Leng Tan, et al. Multi-center validation study of automated classification of pathological slowing in adult scalp electroencephalograms via frequency features. International Journal of Neural Systems, page 2150016, 2021.
[52] Subhrajit Roy, Umar Asif, Jianbin Tang, and Stefan Harrer. Seizure type classification using eeg signals and machine learning: Setting a benchmark. In 2020 IEEE Signal Processing in Medicine and Biology Symposium (SPMB), pages 1-6. IEEE, 2020.
[53] Subhrajit Roy, Isabell Kiral, Mahtab Mirmomeni, Todd Mummert, Alan Braz, Jason Tsay, Jianbin Tang, Umar Asif, Thomas Schaffter, Mehmet Eren Ahsen, et al. Evaluation of artificial intelligence systems for assisting neurologists with fast and accurate annotations of scalp electroencephalography data. EBioMedicine, page 103275, 2021.
[54] R Shantha Selvakumari, M Mahalakshmi, and P Prashalee. Patient-specific seizure detection method using hybrid classifier with optimized electrodes. Journal of medical systems, 43(5):1-7, 2019.
[55] Vinit Shah, Meysam Golmohammadi, Saeedeh Ziyabari, Eva Von Weltin, Iyad Obeid, and Joseph Picone. Optimizing channel selection for seizure detection. In 2017IEEE Signal Processing in Medicine and Biology Symposium (SPMB), pages 1-5. IEEE, 2017.
[56] Vinit Shah, Eva Von Weltin, Silvia Lopez, James Riley McHugh, Lillian Veloso, Meysam Golmohammadi, Iyad Obeid, and Joseph Picone. The temple university hospital seizure detection corpus. Frontiers in neuroinformatics, 12:83, 2018.
[57] Ali Hossam Shoeb. Application of machine learning to epileptic seizure onset detection and treatment. PhD thesis, Massachusetts Institute of Technology, 2009.
[58] Ali H Shoeb and John V Guttag. Application of machine learning to epileptic seizure detection. In ICML, 2010.
[59] Ali Shoeb, Herman Edwards, Jack Connolly, Blaise Bourgeois, S Ted Treves, and John Guttag. Patient-specific seizure onset detection. Epilepsy & Behavior, 5(4):483-498, 2004.
[60] Nishant Sinha, Justin Dauwels, Marcus Kaiser, Sydney S Cash, M Brandon Westover, Yujiang Wang, and Peter N Taylor. Predicting neurosurgical outcomes in focal epilepsy patients using computational modelling. Brain, 140(2):319-332, 2017.
[61] NJ Stevenson, Karoliina Tapani, Leena Lauronen, and Sampsa Vanhatalo. A dataset of neonatal eeg recordings with seizure annotations. Scientific data, 6(1):1-8, 2019.
[62] Andriy Temko, Achintya Sarkar, and Gordon Lightbody. Detection of seizures in intracranial eeg: Upenn and mayo clinic's seizure detection challenge. In 2015 37th Annual International Conference of the IEEE Engineering in Medicine and Biology Society (EMBC), pages 6582-6585. IEEE, 2015.
[63] Prasanth Thangavel, John Thomas, Wei Yan Peh, Jin Jing, Rajamanickam Yuvaraj, Sydney S Cash, Rima Chaudhari, Sagar Karia, Rahul Rathakrishnan, Vinay Saini, et al. Time-frequency decomposition of scalp electroencephalograms improves deep learning-based epilepsy diagnosis. International Journal of Neural Systems, page 2150032, 2021.
[64] Pierre Thodoroff, Joelle Pineau, and Andrew Lim. Learning robust features using deep learning for automatic seizure detection. In Machine learning for healthcare conference, pages 178-190. PMLR, 2016.
[65] John Thomas, Prasanth Thangavel, Wei Yan Peh, Jin Jing, Rajamanickam Yuvaraj, Sydney S Cash, Rima Chaudhari, Sagar Karia, Rahul Rathakrishnan, Vinay Saini, et al. Automated adult epilepsy diagnostic tool based on interictal scalp electroencephalogram characteristics: A six-center study. International Journal of Neural Systems, page 2050074, 2021.
[66] Kostas M Tsiouris, Spiros Konitsiotis, Dimitrios D Koutsouris, and Dimitrios I Fotiadis. Unsupervised seizure detection based on rhythmical activity and spike detection in eeg signals. In 2019 IEEE EMBS International Conference on Biomedical & Health Informatics (BHI), pages 1-4. IEEE, 2019.
[67] André B Valdez, Erin N Hickman, David M Treiman, Kris A Smith, and Peter N Steinmetz. A statistical method for predicting seizure onset zones from human single-neuron recordings. Journal of neural engineering, 10(1):016001, 2012.
[68] Paul Vanabelle, Pierre De Handschutter, Riëm El Tahry, Mohammed Benjelloun, and Mohamed Boukhebouze. Epileptic seizure detection using eeg signals and extreme gradient boosting. Journal of biomedical research, 34(3):228, 2020.
[69] Ashish Vaswani, Noam Shazeer, Niki Parmar, Jakob Uszkoreit, Llion Jones, Aidan N Gomez, Lukasz Kaiser, and Illia Polosukhin. Attention is all you need. In Advances in neural information processing systems, pages 5998-6008, 2017.
[70] Xiaoshuang Wang, Xiulin Wang, Wenya Liu, Zheng Chang, Tommi Karkkainen, and Fengyu Cong. One dimensional convolutional neural networks for seizure onset detection using long-term scalp and intracranial eeg. Neurocomputing, 459:212-222, 2021.
[71] Elizabeth Waterhouse. New horizons in ambulatory electroencephalography. IEEE Engineering in Medicine and Biology Magazine, 22(3):74-80, 2003.
[72] Mengni Zhou, Cheng Tian, Rui Cao, Bin Wang, Yan Niu, Ting Hu, Hao Guo, and Jie Xiang. Epileptic seizure detection based on eeg signals and cnn. Frontiers in neuroinformatics, 12:95, 2018.
[73] Saeedeh Ziyabari, Vinit Shah, Meysam Golmohammadi, Iyad Obeid, and Joseph Picone. Objective evaluation metrics for automatic classification of eeg events. arXiv preprint arXiv:1712.10107, 2017.

What is claimed is:

1. An automated method of seizure detection using a method selected from scalp electroencephalogram (EEG), intracranial EEG (iEEG) and a combination thereof, the method comprising:
(a) applying a transformer with a convolutional neural network (CNN) and a belief matching (BM) loss to acquire subject data from at least one single-channel segment (channel-level), acquiring channel-level data;
(b) extracting from the channel-level data at least two regional features, thereby acquiring data from multi-channel segments (segment level);
(c) from the multi-channel segment data, predicting seizure probability in the multi-channel segments;
(d) repeating (c) at least once, acquiring successive segment-level outputs; and
(e) applying at least one convolutional-based postprocessing module on the successive segment-level outputs, thereby detecting the seizure.

2. The method according to claim 1, wherein the transformer is a deep learning model based on attention mechanism.

3. The method according to claim 2, wherein the attention mechanism is self-attention.

4. The method according to claim 1, wherein loss of order information at the channel-level is mitigated by applying positional encoding to channel-level input data.

5. The method according to claim 1, wherein subject data are acquired from at least 10 channel segments, at least 15 channel segments, or at least 20 channel segments.

6. The method according to claim 1, wherein subject data are acquired from at least 21 channel segments.

7. The method according to claim 1, wherein the channel-level data are data from an electroencephalogram.

8. The method according to claim 1, wherein the channel-level data yields seizure probabilities for each channel.

9. The method according to claim 1, wherein the seizure probabilities are arranged into regions according to the scalp topology: frontal, central, occipital, and parietal. Besides those four local regions, we also define a “global” region containing.

10. The method according to claim 9, wherein the seizure probabilities are further arranged into a “global” region containing all channels.

11. The method according to claim 9, further comprising extracting from each region at least one of seven statistical features: mean, median, standard deviation, maximum value, minimum value, and value at 25% and 75% percentile, thereby forming a feature set.

12. The method according to claim 11, wherein there are five regions and 5×7=35 features are extracted.

13. The method according to claim 11, further comprising computing from all channel-level outputs normalized histogram features (5 bins, range [0,1]) and including them into the feature set, bringing the total features in the feature set to 40.

14. The method according to claim 1, wherein the repeating in (d) is performed for all EEG segments.

15. The method according to claim 1, wherein (e) comprises: applying at least one ID smoothing filter, thereby removing from the data isolated seizure detections (e.g., false positives), and smoothing regions with significant confidence variations, and stabilizing the detections.

16. The method according to claim 1, wherein (e) further comprises: following smoothing, thresholding to the seizure probabilities to round them to zeros (seizure-free) or ones (seizure).

17. The method according to claim 16, wherein a threshold value θ∈{0.1, 0.2, 0.3, 0.4. 0.5, 0.6. 07, 0.8, or 0.9} is utilized.

18. The method according to claim 17, further comprising: following thresholding, identifying consecutive is of length smaller than Nc, and replacing the identified 1 s with 0 s, thereby removing short detections, leading to fewer false positives and more false negatives, as the system may miss short seizures.

19. The method according to claim 18, wherein Nc∈{1, 2, 3, 4, 5, 6, 7, 8. 9. 20, 11, 12, 13, 14, 15, 16, 17, 18, 19, or 20}.

20. The method according to claim 18, wherein following the replacing remaining sequences of consecutive is are identified, and the start and end time of the consecutive is is identified, thereby resulting in a final output of the EEG-level seizure detector being the start and end times of the detected seizures.
